(19) United States
(12) Patent Application Publication
(10) Pub. No.: US 2006/0127365 A1
(43) Pub. Date: Jun. 15, 2006

(54) Method of cell therapy using fused cell hybrids

(76) Inventors: Victor Nurcombe, Chapel Hill, Queensland (AU); David Robert James Monaghan, Bocany, New South Wales (AU); Douglas Hamish Campbell, Drumoyne, New South Wales (AU); John David Priest, Balmain, New South Wales (AU); Alan Douglas Watts, Dee Why, New South Wales (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/515,579

(22) PCT Filed: May 30, 2003
(86) PCT No.: PCT/AU03/00666

§ 371 Date: Oct. 31, 2005

Related U.S. Application Data
(60) Provisional application No. 60/384,882, filed on May 31, 2002.
(60) Provisional application No. 60/387,284, filed on Jun. 7, 2002.

(30) Foreign Application Priority Data
Jun. 19, 2002 (AU) PS 3078
Sep. 5, 2002 (AU) 2002951222
Sep. 5, 2002 (AU) 2002951223

(51) Int. Cl. A61K 35/30; C12N 5/08

(52) U.S. Cl. 424/93.7; 435/368

(57) ABSTRACT

The present invention relates generally to the field of tissue engineering and more particularly to a method for generating tissue suitable for use in tissue replacement and/or tissue rejuvenation therapy and/or as a source of cell-derived therapeutic or diagnostic agents including proteins and hormones. Even more particularly, the present invention contemplates the use of cell fusion techniques involving single cell, mini-bulk or macro-bulk cell fusion to generate tissue or cells useful for tissue replacement and/or tissue rejuvenation therapy or a range of organs or areas of the body. The resulting tissue or cells may also secrete or generate a range of cytokines, enzymes, hormones and the like which have improved or more efficacious properties relative to analogous molecules produced from non-fused cells. The present invention further provides an apparatus having aspects controlled by data processing means which facilitates the fusion of a pair of cells. Of the pair of cells, at least one of the cells in the pair may be a mature cell or is capable of differentiating or developing into a mature cell. The subject invention further provides isolated molecules such as cytokines, receptors, antibodies, hormones, heat shock proteins, enzymes, and glycoproteins such as mucins, lectins and heparan sulfates derived from fused cells. These molecules may be characterized by having altered glycosylation patterns, altered post-translational modifications, greater activity, being more efficacious or being more stable relative to analogous molecules from non-fused cells. The present invention further provides novel cell fusates or cell hybrids having a pattern of cell surface markers unique relative to the at least two cells which fuse together to generate the cell. These cell markers are useful in selecting particular cell hybrids and as proprietary tags.

### BACKGROUND OF THE INVENTION

1. Field of the Invention

The present invention relates generally to the field of tissue engineering and more particularly to a method for generating tissue suitable for use in tissue replacement and/or tissue rejuvenation therapy and/or as a source of cell-derived therapeutic or diagnostic agents including proteins and hormones. Even more particularly, the present invention contemplates the use of cell fusion techniques involving single cell, mini-bulk or macro-bulk cell fusion to generate tissue or cells useful for tissue replacement and/or tissue rejuvenation therapy or a range of organs or areas of the body. The resulting tissue or cells may also secrete or generate a range of cytokines, enzymes, hormones and the like which have improved or more efficacious properties relative to analogous molecules produced from non-fused cells. The present invention further provides an apparatus having aspects controlled by data processing means which facilitates the fusion of a pair of cells. Of the pair of cells, at least one of the cells in the pair may be a mature cell or is capable of differentiating or developing into a mature cell. The subject invention further provides isolated molecules such as cytokines, receptors, antibodies, hormones, heat shock proteins, enzymes, and glycoproteins such as mucins, lectins and heparan sulfates derived from fused cells. These molecules may be characterized by having altered glycosylation patterns, altered post-translational modifications, greater activity, being more efficacious or being more stable relative to analogous molecules from non-fused cells. The present invention further provides novel cell fusates or cell hybrids having a pattern of cell surface markers unique relative to the at least two cells which fuse together to generate the cell. These cell markers are useful in selecting particular cell hybrids and as proprietary tags.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Conventional therapies include the identification of chemical or proteinaceous molecules which are aimed at ameliorating physiological conditions which have developed in a patient or non-human subject. Frequently, the physiological conditions arise from degenerative disorders resulting in cell apoptosis, autoinmmune responses and altered genetic and metabolic networks. Many of these types of conditions can be treated at the symptomatic level but long term repair or permanent amelioration of the condition is generally not possible.

This is certainly the case with, for example, autoimnnune conditions. Such conditions include multiple sclerosis, rheumatoid arthritis and insulin-dependent diabetes mellitus (IDDM). The immune system in general can frequently exacerbate adverse physiological conditions such as having hay fever, asthma and anaphylactic shock.

IDDM results from the selective destruction of insulin-producing β-cells in the islets of the pancreas, within an autoinmmune inflammatory “insulitis” lesion (Honeyman et al., Springer Semin. Immunopathol. 14: 253-274, 1993; Bach, Endocrine Rev. 15: 516-542, 1994). This is also referred to as type I diabetes. Previously, target autoantigens which trigger or drive immune reactivity to β-cells have been considered as potential targets for diagnostic applications and also as agents or targets for specific immunotherapy (Adorini et al., Springer Semn. Immunopathol. 14: 187-199, 1992; Muir et al., Diabetes/Metab. Review 9: 279-287, 1993; Harrison, Mol. Med. 1: 722-727, 1995).

Despite the availability of drugs to ameliorate many of the symptoms of degenerative disorders, where cell apoptosis is occurring or where metabolic and neurological networks are permanently damaged, such as with neurodegenerative disorders, alternative protocols are needed to treat affected subjects.

Alternative protocols need, therefore, to be considered.

One such alternative strategy is tissue replacement therapy including organ transplantation. The latter can be effective but suffers from low availability of suitable replacement tissue and the possibility-of rejection in the absence of immune suppression drugs and/or suitable graft tissue which reduces the likelihood of rejection.

In work leading up to the present invention, the inventors proposed to use cell fusion technology to generate cells or tissue comprising for use in tissue replacement and/or rejuvenation therapy. Cell fusion has been rendered possible through chemical, biological and physical means. Examples of these techniques include polyethylene glycol (PEG) fusion, fusagenic virus fusion and electrofusion, respectively.

Cell-lines can be immortal, enabling them to replicate indefinitely, as opposed to being metabolically active but unable to divide. The immortality is due to genetic alterations, such as loss of tumor suppressor genes. Conversely, a primary cell normally has limited proliferative capacity.

In most cases, the goal of cell-cell fusion is to endow the properties of a cell line, which lives eternally, to an isolated primary cell, such that the properties of this primary cell are preserved indefinitely by virtue of its newly created ability to proliferate forever like a cell line. Generally, this technique has been applied to the generation of monoclonal antibodies producing B-cell hybridomas.

One commonly used technique is chemical fusion using, for example, PEG. This technology has been particularly successful in generating hybridomas. In a typical PEG-mediated fusion of 100 million spleen cells and 10 million myeloma cells, the yield of fused cells would be less than 1000, of which four or five cloned cells, but frequently none, make antibody of the desired specificity. This leads to the practice of doing 5-10 fusion operations at one time, resulting in laborious subsequent processing to find the suitable hybridoma.

The fusion probability can be improved by exposure of the cells to intense electric fields for very brief periods; this process is known as “electroporation”, for example, see Zimmerman, Biochimica et Biophysica Acta 694: 227-278, 1982. The fusion probability for cells in suspension, however, remains low, for example, 1.5×10⁻⁵ (see U.S. Pat. No. 4,832,814).

A higher success rate can be achieved using chemical agents to effect linkage and proximation of cell pairs of the desired type (i.e. myeloma and B cell), in a suspension prior to electric field exposure. The method has not found extensive use, perhaps because of its complexity, susceptibility to contamination or low efficacy. This is especially significant in cases where a molecule occurring at low incidence on a cell's surface is the antigen of interest in immunization and fusion. Furthermore, the fusion process between the cells is indiscriminate.

Electrofusion of cells involves bringing cells together in close proximity and exposing them to an alternating electric field. Under appropriate conditions, the cells are pushed together and there is a fusion of cell membranes and then the formation of fusate cells or hybrid cells. Electrofusion of cells and apparatus for performing same are described in, for example, U.S. Pat. Nos. 4,441,972, 4,578,168 and 5,283,194, International Patent Application No. PCT/AU92/00473 [WO 93/05166], Pohl, “Dielectrophoresis”, Cambridge University Press, 1978 and Zimmerman et al., Biochimica et Bioplzysica Acta 641: 160-165, 1981.

Whilst electrofusion has had some limited success, for example in developing hybridomas, low rates of fusions and difficulties with fusion machines and in culturing the resulting hybrid cells have limited the use of the technology.

In relation to tissue replacement and rejuvenation therapy, one problem is providing sufficient cells from a subject or a histocompatible (i.e. HLA-matched) subject for use in replacement therapy.

Stem cells have the potential for being progenitors to a range of differentiated cell types. Adult stem cells are the pluripotent cells of the body, being progenitors to many cell types and awaiting specific signals to turn into a given tissue in the body. Adult stem cells are, to a greater or lesser extent, committed to a particular differentiation pathway. Possibly, bone marrow-derived mesenchymal stem cells are one of the most pluripotent of the adult stem cells and are capable of differentiating into such diverse cell types such as cardiomyocytes and endothelial cells.

Embryonic stem (ES) cells are the most pluripotent stem cells of all, having the ability to make any cell or tissue in the body. One of the great challenges for developmental biologists has been the ability to control the direction in which ES cells grow and differentiate.

There is a desire to control ES cell differentiation. However, this is presently partially accomplished in a somewhat haphazard fashion with mixed results using cocktails of one or more growth factors and/or cytokines. Alternatively, stem cells are sometimes injected directly into damaged tissue and a reliance is placed on the cellular microenvironment of the target tissue to control the fate of differentiation of the injected stem cells.

In accordance with the present invention, the inventors have now combined cell fusion technology and stem cell technology to enable the generation of large amounts of cells having a potential for use in tissue replacement and/or regeneration therapy. The resulting fusates are also proposed to be useful as a source of cellular-derived molecules such as cytokines, enzymes, heat shock proteins, hormones and even ligands for cell receptors.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention relates generally to cell fusion where at least one cell in the fusion process is a stem cell.

Stem cells can be categorized into the following classifications. All categories of stem cells are referred to herein as “stem cells”. The most pluripotent and earliest in terms of developmental stage, are the “embryonic stem (ES) cells” or “ES cells”. ES cells may be freshly derived primary cells, an ES cell-line, or an embyronic carcinoma (EC) cell line. All other stem cells from somatic tissue (every tissue excluding germ cell tissue) are defined in general terms as “somatic stem cells”, but might be commonly known as any or all of the following: “adult stem cells”, “mature stem cells”, “progenitor cells”, “progenitor stem cells”, “precursor cells” and “precursor stem cells”. The other class of non-ES cell is defined as “germ line stem cells”. Finally, non-stem cells are herein described as “mature cells”, but are also known as “differentiated cells”, “mature differentiated cells”, “terminally differentiated cells” and “somatic cells”. Mature cells may also be primary isolated cells derived from tissue or an immortal cell line or a tumor-derived cell-line. The present invention further encompasses “precursor forms of a mature cell” which includes all cells that do not fulfil commonly used scientific definitions for either stem cells or mature cells.

The present invention provides a method for the generation of cells for use in cell replacement and/or rejuvenation therapy. The cells may also be a source of molecules such as proteins, hormones, cytokines, enzymes, heat shock proteins and ligands for cell receptors. The method involves the fusion of a pair of cells wherein at least one cell is a stem cell (either embryonic or somatic) and the other is a somatic stem cell, a precursor form of a mature cell, or a mature cell. The method also includes so-called “autofusion”, which is defined as the fusion of two cells of the same cell type. For example, the method includes the fusion of ES cells with ES cells. The selection of the mature cell depends on the condition being treated such as replacing or rejuvenating traumatized tissue, cancerous tissue or specific cells which have an impaired capacity to generate a particular product. In a preferred embodiment, one of the pair of cells is an embryonic stem (ES) cell and the other of the pair is a somatic stem cell. In another embodiment, one of the pair of cells is an ES cell and the other of the pair is a mature cell. Still yet another embodiment provides fusion between a neural stem cell and an ES cell from an adult or an embryo or embryonic tissue. The stem cells may be derived from an embryo or at a post-embryo/pre-adult stage or from any of the brain, intestinal epithelium, epidermis, skin, pancreas, kidney, liver, breast, lung, muscle, heart, eye, bone marrow, spleen and/or the immune system.

Accordingly, by combining the proliferative capacity of one type of highly pluripotent stem cell, such as as an ES cell, and the differentiation pathway commitment of an adult stem cell or mature cell, in a fused cell hybrid of the two, a therapeutic cell-type can be delivered which can be both expanded in larger numbers required for tissue therapy, and be differentiated into the target tissue so desired.

In yet another embodiment, one or both of the pair of cells, is carrying a transgene encoding a gene product which controls the fate of the resulting fusate. An example of this is a myosin heavy chain gene or cDNA, which may control the fate of the resulting fusate down the cardiac lineage. The transgene may be present in the cell in either a stably integrated fashion, or a transient fashion. Stable integration would constitute the transgene becoming part of one or more host cell chromosomes. The transgene is present in a mammalian cell expression vector, such as a plasmid, or a virus, such as an adenovirus or a lentivirus. Alternatively, the transgene may not encode a protein, but rather antisense RNA, or alternatively, interference RNA (iRNA). The iRNA in this way would be able to selectively switch on or off genes involved in the fate of the stem cell, and also the resulting fusate. The transgene may be either constitutive or under inducible regulatory control. In a preferred embodiment, expression of the transgene keeps the stem cells and fusates in an undifferentiated state indefinitely but switched to allow differentiation following fusion, and subsequent expansion in a culture system.

Generally, the method involves selecting the cells and positioning them in a fluid-filled chamber adopted for use as a cell-fusing chamber. Individual pairs of cells may be involved in the fusion process, i.e. single cell fusion, or bulk fusion may occur with two populations each comprising two or more cells. Bulk fusion may be mini-bulk fusion where from about 2 to about 1000 cells are involved or macro-bulk fusion where greater than about 1000 cells are involved. Fusion may be facilitated by chemical means such as in the presence of PEG, biological means, such as in the presence of a fusagenic virus or by electrical means, i.e. electrofusion. The fusion may also involve a combination of these techniques. The cells may also be treated with a cytokine such as interleukin 3 (IL-3) to facilitate fusion.

Following cell fusion, a fused cell (fusate cell) or otherwise known as a hybrid cell is obtained comprising of nuclei of at least two cells encased in a fused lipid bilayer from the cells involved in the fusion. The nuclei of the cells fuse resulting in a hybrid cell with an abnormal number of chromosomes, which might be quadraploid or containing less or a greater number of chromosomes. The hybrid cell has the ability to divide and proliferate under appropriate culture conditions.

In a preferred embodiment, the fusion is by electrical means and involves single cell fusion although the present invention clearly extends to mini-bulk and macro-bulk electrofusion Of mini-bulk and macro-bulk techniques, mini-bulk electrofusion is the preferred embodiment as it has clear advantages over macro-bulk due to the small number of ES and somatic stem cells likely to be isolated for individual experiments. The mini-bulk method contains specialized methods for dealing with the smaller numbers of cells pre-fusion, post-fusion and during the course of fusion.

In single cell electrofusion, an electrical current having a pre-determined waveform to the electrodes is then applied, which permits the cells to fuse. Preferably, the cells to be fused are selected from separate containers using a pipette and positioned in the fusing chamber such that the cells are substantially located between but separate from both electrodes. The pipettes are conveniently operated via a drive system adapted to move the pipette with respect to the containers separately holding the cells and the fusing chamber. The operation of the drive system may also be controlled by data processing means which is adapted to receive and process input commands from the user. The pipettes are most convenient for use in single cell fusions and in which case, the preferred form of pipette is a micro-pipette.

Generally, once fusion has occurred, the resulting hybrid cell is recovered in a suitable rich medium prior to being expanded in culture for use in tissue replacement, rejuvenation therapy and cellular therapeutics. The recovery medium should contain factors allowing the recovery of the cell fusate following the stress of fusion. Such a supplement could include a high percentage of fetal calf serum, for example 20%.

The present invention provides, therefore, a method for the prophylaxis or treatment of trauma or disease in a subject, the method comprising expanding a culture comprising a hybrid cell generated between a pair of cells wherein one of the cells is a stem cell (either embryonic or somatic) and another of the cells is a somatic stem cell, a precursor form of a mature cell or a mature cell thereof fused together by fusion and introducing the expanded culture of cells to the subject. The present invention further contemplates autofusion between two cells of the same type.

The hybrid cells generated via cell fusion-may comprise unique cell surface markers which are useful in selecting these cells, monitoring their status and movement in a subject and as proprietary tags. Furthermore, the present invention provides novel metabolites including cytokines, heat shock proteins, hormones, ligands for receptors and enzymes produced by the fused cells. The fusates are, therefore, also useful in cellular therapeutics.

The present invention provides, in a further embodiment, a system of cell fusion comprising:
(i) selecting two populations of cells, each population comprising one or more cells, to fuse together;
(ii) fusing the at least two cells together;
(iii) subjecting the fused cells to culture conditions to grow the fused cells;
(iv) optionally subjecting the cells to data processing means to analyze cell surface markers and/or secretion or generation of protein or non-protein metabolites; and
(v) providing cultures of fused cells to other entities.

The fused cells may also be used for further fusion experiments, i.e. refusion to other cells, in a continuous process of “cell-breeding”.

This aspect of the present invention covers a method of contract research where entities either provide cell populations for fusion together and/or who seek to exploit cells which have been fused together. Such entities include inter alia patients, medical practitioners, a pharmaceutical entity or a researcher.

### BRIEF DESCRIPTION OF THE FIGURES

FIG. 1 is a block diagram of an example of apparatus for fusing cells.

FIG. 2 is a schematic diagram of the apparatus of FIG. 1.

FIG. 3 is a schematic diagram of the pipette of FIG. 1.

FIG. 4 is a flow chart of an overview of the process of fusing cells using the apparatus of FIG. 1.

FIGS. 5A to 5C are a flow chart of the process of fusing cells implemented by the apparatus of FIG. 1.

FIGS. 6A and 6B are schematic diagrams of cells being drawn into and expelled from the pipette of FIG. 3.

FIGS. 6C and 6D are schematic diagrams of the arrangement of the electrodes and cells in the fusion well during operation of the apparatus of FIG. 1.

FIGS. 7A to 7G are examples of pulse sequences that may be used in the apparatus of FIG. 1.

FIG. 8A is a schematic plan view of a second example of apparatus for fusing cells.

FIG. 8B is a schematic side view of the modified well array of FIG. 8A.

FIG. 9A is a schematic plan view of a third example of apparatus for fusing cells.

FIG. 9B is a schematic side view of one of the cells shown in FIG. 9A.

FIG. 9C is a schematic perspective view of the first electrodes of FIG. 9.

FIG. 10 is a schematic diagram of the pipette of FIG. 3 modified to include an electrode.

FIG. 11 is a block diagram of a modified version of the apparatus of FIG. 1 adapted to use two of the pipettes shown in FIG. 10.

FIG. 12 is a schematic diagram of the apparatus of FIG. 11.

FIG. 13A is a schematic diagram of the pipette of FIG. 10 modified to include a radiation source.

FIG. 13B is a schematic diagram of the pipette of FIG. 3 modified to include an alternative radiation source.

FIG. 14 is a schematic diagram of the pipette of FIG. 3 retrieving a number of cells.

FIG. 15 is a schematic diagram of the pipettes of FIG. 11 positioning cells for subsequent fusion.

FIG. 16 is a schematic diagram of the pipettes of FIG. 11 and fused cells.

FIG. 17 is a schematic diagram of the pipette of FIG. 3 modified to include a radiation source.

FIG. 18A is a schematic diagram of the pipette of FIG. 3 with an alternative actuator.

FIG. 18B is a schematic diagram of the operation of the actuator of FIG. 18A.

FIG. 18C is a schematic diagram of a first example of the pipette of FIG. 18A modified for use with a bladder.

FIG. 18D is a schematic diagram of a second example of the pipette of FIG. 18A modified for use with a bladder.

FIG. 19 is a schematic diagram of a cutting tool used for cutting cells.

FIG. 20 is a block diagram of an example of apparatus for automatically fusing cells.

FIG. 21 is a schematic diagram of the apparatus of FIG. 20.

### DETAILED DESCRIPTION OF THE INVENTION

The present invention provides a means for generating tissue suitable for use in replacement, rejuvenation therapy and as a source of cellular therapeutics. More particularly, the present invention contemplates a method for generating mature cells or cells with a propensity to develop or differentiate into mature cells for use in the treatment or prophylaxis of mature trauma or disease.

Accordingly, one aspect of the present invention provides a method for generating mature cells or cells capable of differentiating into mature tissue, said method comprising selecting first and second populations of cells and positioning said first and second populations of cells in a fluid-filled fusing chamber and then subjecting said populations of cells to conditions which facilitate fusion to occur between at least one pair of cells.

Reference to “cells capable of differentiating into mature tissue” includes the capacity in vivo, in vitro or ex vivo. The term “in vivo” means after introduction of the cells to a patient. The term “in vitro” means in cell culture. In any case, additional factors may be introduced to facilitate differentiation and/or development into the desired cell types. The term “ex vivo” includes differentiating tissue in culture systems or a suitable host animal such as a pig to produce all or part of an organ or tissue or a pure population of cells.

Reference to a population of cells includes reference to a single cell or a multiplicity of cells. Accordingly, this aspect of the present invention extends to both single cell fusion between a pair of cells (i.e. each population of cells comprises a single cell) as well as bulk cell fusion between two populations of cells where each population comprises at least two cells. Bulk fusion may be mini-bulk fusion (involving from about 2 to about 1000 cells) and macro-bulk fusion (involving approximately greater than 1000 cells).

The cells within each population of cells may be each selected from a range of different types of cells. Examples of populations of cells between which fusion is to be induced or otherwise facilitated include embryonic stem (ES) cells and stem cells from an adult; ES cells and mature cells from an adult; ES cells and stem cells from the post-embryo/pre-adult stage; ES cells and cells from the post-embryo/pre-adult stage; neural stem cells from an adult and stem cells; neural stem cells from an adult and mature cells; neural stem cells from a post-embryo/pre-adult stage including adult neural stem cells.

A suitable list of stem and mature cells and their application for use in transplant and rejuvenation therapy is shown in Table 1. All such stem and mature cells are contemplated and are encompassed by the present invention. As indicated in Table 1, a mature cell may be derived from any human tissue such as from the brain, epidermis, skin, pancreas, kidney, liver, breast, lung, muscle, heart, eye, bone, gastrointestinal tract, spleen or the immune system. Cells of the immune system include CD4+ T-cells, CD8+ T-cells, NK cells, monocytes, macrophages, dendritic cells and B-cells. It should be noted that the present invention contemplates the fusion of stem cells and mature cells from any source such as a mammal (including human), non-mammalian animal and avian species.

TABLE 1

| Cell type | Application |
|---|---|
| General Stem cell types | |
| Embryonic stem cells | Generation of any tissue for transplant |
| Somatic stem cells | Generation of tissue for transplant |
| Germ stem cells | Generation of tissue for transplant |
| Human embryonic stem cells | Generation of wide variety of tissue for transplant |
| Human epidermal stem cells | Generation of tissue for transplant |

Tissue-specific cells: Includes both somatic stem cells, mature cells and germ line cells
Brain

| | |
|---|---|
| Adult neural stem cells | Generation of neural tissue for transplant |
| Human neurons | Generation of neural tissue for transplant |
| Human oligodendrocytes | Generation of neural tissue for transplant |
| Human astrocytes | Generation of neural tissue for transplant |
| Epidermis | |
| Human keratinocyte stem cells | Generation of epidermal type tissues such as hair |
| | follicles, sebaceous glands and skin for transplant |
| Human keratinocyte transient | Generation of epidermal type tissues such as hair |
| amplifying cells | follicles, sebaceous glands and skin for transplant |
| Human melanocyte stem cells | Generation of epidermal type tissues for transplant |
| Human melanocytes | Generation of epidermal type tissues for transplant |
| Skin | |
| Human foreskin fibroblasts | Generation of skin for transplant |
| Pancreas | |
| Human duct cells | Generation of insulin-producing cells for transplant |
| Human pancreatic islets | Generation of insulin-producing cells for transplant |
| Human pancreatic β-cells | Generation of insulin-producing cells for transplant |
| Kidney | |
| Human adult renal stem cells | Generation of kidney tissue for transplant |
| Human embryonic renal | Generation of kidney tissue for transplant |
| epithelial stem cells | |
| Human kidney epithelial cells | Generation of kidney tissue for transplant |
| Liver | |
| Human hepatic oval cells | Generation of insulin-producing cells for transplant |
| Human hepatocytes | Generation of liver tissue for transplant |
| Human bile duct epithelial cells | Generation of liver tissue for transplant |
| Human embryonic endodermal | Generation of liver tissue for transplant |
| stem cells | |
| Human adult hepatocyte stem | Generation of liver tissue for transplant |
| cells (controversial as to | |
| existence) | |
| Breast | |
| Human mammary epithelial | Generation of mammary (breast) tissue for transplant |
| stem cells | |
| Lung | |
| Bone marrow-derived stem cells | Generation of tissue for transplant including muscle, |
| | cartilage, bone, liver, heart, brain, intestine and lung |
| Human lung fibroblasts | Generation of tissue for transplant including muscle, |
| | cartilage, bone, liver, heart, brain, intestine and lung |
| Human bronchial epithelial cells | Generation of tissue for transplant including muscle, |
| | cartilage, bone, liver, heart, brain, intestine and lung |
| Human alveolar type II | Generation of tissue for transplant including muscle, |
| pneumocytes | cartilage, bone, liver, heart, brain, intestine and lung |
| Muscle | |
| Human skeletal muscle stem | Generation of tissue for transplant |
| cells (satellite cells) | |
| Heart | |
| Human cardiomyocytes | Generation of heart tissue for transplant |
| Bone marrow mesenchymal | Generation of heart tissue for transplant |
| stem cells | |
| Simple Squamous Epithelial | Generation of heart and vascular tissue, for example |
| cells | rebuilding aortic arteries after aneurysm repairs |
| Descending Aortic Endothelial | Generation of heart and vascular tissue, for example |
| cells | rebuilding aortic arteries after aneurysm repairs |
| Aortic Arch Endothelial cells | Generation of heart and vascular tissue, for example |
| | rebuilding aortic arteries after aneurysm repairs |
| Aortic Smooth Muscle cells | Generation of heart and vascular tissue, for example |
| | rebuilding aortic arteries after aneurysm repairs |
| Eye | |
| Limbal stem cells | Regeneration of the entire corneal epithelium for |
| | transplant |
| Corneal epithelial cells | Regeneration of the entire corneal epithelium for |
| | transplant |

Bone Marrow
(in some cases be substituted for cord blood and peripheral blood as a source of some
of the below stem cells)

| | |
|---|---|
| CD34+ hematopoietic stem cells | Generation of a wide variety of tissues for transplant, |
| | including, but not limited to, immune tissue |
| Mesenchymal stem cells | Generation of a wide variety of tissues for transplant, |
| | including, but not limited to, cardiac tissue, bone, |
| | cartilage, muscle, tendon, endothelial tissue, vascular |
| | tissue and neural tissue |
| Osteoblasts (precursor is | Generation of bone for transplant |
| mesenchymal stem cell) | |
| Peripheral blood mononuclear | Generation of a wide variety of tissues for transplant, |
| progenitor cells (hematopoietic | including but not limited to cardiac tissue, bone, |
| stem cells) | cartilage, muscle, tendon, endothelial tissue, vascular |
| | tissue and neural tissue |
| Osteoclasts (precursor is above | Generation of bone for transplant |
| cell type) | |
| Stromal cells | Generation of a wide variety of tissues for transplant, |
| | including but not limited to cardiac tissue, bone, |
| | cartilage, muscle, tendon, endothelial tissue, vascular |
| | tissue and neural tissue |
| Spleen | |
| Human splenic precursor stem | Generation of spleen tissue for transplant |
| cells | |
| Human splenocytes | Generation of spleen tissue for transplant |
| Immnune cells | |
| Human CD4+ T-cells | Generation of immune cells/tissue for transplant |
| Human CD8+ T-cells | Generation of immune cells/tissue for transplant |
| Human NK cells | Generation of immune cells/tissue for transplant |
| Human monocytes | Generation of immune cells/tissue for transplant |
| Human macrophages | Generation of immune cells/tissue for transplant |
| Human dendritic cells | Generation of immune cells/tissue for transplant |
| Human B-cells | Generation of immune cells/tissue for transplant |
| Nose | |
| Goblet cells (mucus secreting | Generation of cells/tissue for sinus tissue repair |
| cells of the nose) | |
| Pseudostriated ciliated columnar | Generation of cells/tissue for sinus tissue |
| cells (located below olfactory | repair/replacement |
| region in the nose | |
| Pseudostriated ciliated | Generation of cells/tissue for sinus tissue |
| epithelium (cells that line the | repair/replacement |
| nasopharangeal tubes) | |
| Trachea | |
| Stratified Epithelial cells (cells | Generation of cells/tissue for trachea |
| that line and structure the | repair/replacement |
| trachea) | |
| Ciliated Columnar cells (cells | Generation of cells/tissue for trachea |
| that line and structure the | repair/replacement |
| trachea) | |
| Goblet cells (cells that line and | Generation of cells/tissue for trachea |
| structure the trachea) | repair/replacement |
| Basal cells (cells that line and | Generation of cells/tissue for trachea |
| structure the trachea) | repair/replacement |
| Oesophagus | |
| Cricopharyngeus muscle cells | Generation of cells/tissue for oesophagus |
| | repair/replacement |
| Oesophageal stem cells | Generation of cells/tissue for oesophagus |
| | repair/replacement |
| Oesophageal transit amplifying | Generation of cells/tissue for oesophagus |
| cells | repair/replacement |
| Reproduction | |
| Female primary follicles | Generation of natural fertility |
| Male spermatogonium | Generation of natural fertility |

In terms of using the cells for tissue replacement therapy or augmentation therapy, at least one population of cells may come from the patient to be treated or from a histocompatibility matched subject (i.e. HLA-matched). Furthermore, at birth, subjects may store cells or tissue for subject use in later life. Such tissue would include placenta tissue, umbilical chord tissue, foreskin, blood or other uteric tissue associated with a fetus.

In general, the preferred fusion is between two cells where one cell is an immature stem cell such as ES cell, neural stem cell, astrocyte or bone marrow mesenchymal-derived stem cell and the other of the cells is a somatic stem cell or a mature differentiated cell. Preferably, stem and mature cells are those listed in Table 1.

Accordingly, another aspect of the present invention contemplates a method for generating mature cells or cells capable of differentiating into mature tissue, said method comprising selecting first and second populations of cells from a first population comprising an immature stem cell selected from a stem cell listed in Table 1 from an embryo, post-embryo/pre-adult or adult and from a second population comprising an adult stem cell or mature differentiated cell selected from the list in Table 1, positioning said first and second populations of cells in a fluid-filled fusing chamber and then subjecting said populations of cells to conditions which facilitate fusion to occur between at least one pair of cells.

The method may further comprise collecting the fused cells and subjecting same to culture conditions to facilitate expansion or proliferation to generate sufficient tissue for use in transplantation or regeneration.

The conditions which facilitate fusion include electrical conditions (e.g. electrofusion, electroporation), chemical conditions (e.g. PEG fusion) and biological conditions (e.g. a fusagenic virus) or a combination of any two or more of the above. Molecules such as cytokines (e.g. IL-3) may also be used to facilitate fusion between cells. Bulk fusion encompasses mini-bulk and bulk-macro fusion.

In all cases, fusion may be by single cell fusion or bulk fusion.

The preferred form of chemical fusion is via PEG. PEG fusion may be conveniently accomplished by the method described in U.S. Pat. No. 4,832,814 and may optionally be used in combination with electroporation.

According to this embodiment, there is provided a method for generating mature cells or cells capable of differentiating into mature tissue, said method comprising selecting first and second populations of cells from a first population comprising a stem cell listed in Table 1 from an embryo, post-embryo/pre-adult or adult and from a second population comprising a somatic stem cell or mature cell selected from the list in Table 1, positioning said first and second populations of cells in a fluid-filled fusing chamber and then subjecting said populations of cells to chemical conditions which facilitate fusion to occur at least between one pair of cells.

Preferably, chemical fusion is conducted under bulk fusion conditions.

Preferably, the first population comprises ES cells.

The most preferred form of fusion, however, is electrofusion. Electrofusion may be conducted under single cell or bulk fusion conditions although single cell fusion is preferred.

Accordingly, another aspect of the present invention provides a method for generating mature cells or cells capable of differentiating into mature tissue, said method comprising selecting first and second populations of cells from a first population comprising a stem cell listed in Table 1 from an embryo, post-embryo/pre-adult or adult and from a second population comprising an adult stem cell or mature differentiated cell selected from the list in Table 1, positioning said first and second populations of cells in a fluid-filled fusing chamber and then subjecting said populations of cells to electrofusion conditions to facilitate fusion of at least one pair of cells.

Insofar as single cell fusion is concerned, the present invention is directed to a method for generating mature cells or cells capable of differentiating into mature tissue, said method comprising selecting first and second populations of cells and positioning first and second populations of cells between two electrodes in a fluid-filled fusing chamber and then applying an electrical current having a predetermined waveform to the electrode to induce at least one pair of cells to fuse.

Preferably, the cells are maintained in the fusing chamber close to but separate from the electrodes. Consequently, the cells are held separate from each electrode.

Preferably, electrofusion is conducted using a single pair of cells wherein at least one cell in the pair is a stem cell (e.g. an ES cell, neural stem cell, astrocyte or bone marrow stem cell) or another stem cell as listed in Table 1 and the other cell is the pair in a mature cell or a precursor form thereof such as from the brain, gastrointestinal tract, epidermis, skin, pancreas, kidney, liver, breast, lung, muscle heart, eye, bone marrow, spleen or the immune system. Examples of such cells are listed in Table 1. Furthermore, in a preferred embodiment, the pancreatic tissue generated is used for tissue replacement or tissue rejuvenation therapy.

According to this embodiment, there is provided a method for generating mature cells or cells capable of differentiating into mature tissue for use in tissue replacement and/or rejuvenation therapy and/or as a source of cell-derived molecules, said method comprising selecting first or second cells wherein one of said first or second cells is a stem cell selected from the list in Table 1 and the other of said first and second cells is a mature cell or a stem cell thereof selected from the list in Table 1 and positioning said first and second cells between two electrodes in a fluid-filled fusing chamber and then applying an electrical current having a predetermined waveform to the electrode to induce the cells to fuse, and then culturing the said fused cells under conditions to permit expansion of the cells.

Regardless of the mode of fusion, reference to “expansion” of the cells includes inducing proliferation of the original hybrid cell to a number of cells sufficient to implant into a subject. Expansion of the cell culture may be by chemical induced proliferation including growth factor-mediated proliferation. Furthermore, cells may be differentiating tissue in culture systems to produce all or part of an organ or target tissue or a pure population of mature cells.

In relation to expanding tissue for transplantation and/or tissue regeneration, the fused cells may also be cultured in the presence of agents such as growth factors or cytokines or small molecule chemicals.

Furthermore, the resulting hybrid cells may have a unique set of surface markers. DNA, RNA or antibody microarray technology may be used to identify the surface markers and in particular growth factor receptors. Such growth factors include Bone Marrow Morphogenetic Proteins) BMPs, cytokines (e.g. G-CSF, GM-CSF, TNF-α, TNF-β, LIF), interleukins (e.g. IL-1, 2, 3, 4, 5, 6, 7, 8, 9, 10 and 11), and Ciliary Neurotrophic Factor (CNTF) amongst many others. The identification of growth factor receptors provides a means of inducing proliferation such as by culturing the cells in vitro in the presence of the growth factors and/or administering the hybrid cells together with or sequential to (in either order) the growth factor.

The identification of a novel spectrum of surface markers is also useful in order to select particular hybrid cells and to act as proprietary tags.

The present invention provides, therefore, a data set of surface markers of a hybrid cell formed by fusing a stem cell with a mature cell or precursor thereof, said data set defining said hybrid cell.

The stem cell may be any form of progenitor cell including an embryonic stem (ES) cell or adult or immature non-stem cell. The latter two types of stem cells are particularly useful due to their ability to generate pluripotent cell lines which can be expanded under certain culture conditions and/or in the presence of a proliferating agent.

The ES cells may be obtained from a variety of sources and may be primary isolated ES cells or an artificially or naturally created ES cell line. Similarly, the neural stem cells may be isolated such as from a spinal tap or other suitable location and be either a naturally occurring or artificially created cell line. Other stem cells may be isolated by a variety of techniques including biopsies and bone marrow extractions.

The ES cells, neural stem cells or other stem cells may also be first genetically modified to introduce particularly useful traits such as enhanced production of therapeutically useful cytokines or engineered to produce receptors to help target the cells to appropriate locations in the pancreas, brain, heart or other tissues of the body.

Accordingly, another aspect of the present invention contemplates a method for generating mature cells or cells capable of differentiating into mature tissue for use in tissue replacement and/or rejuvenation therapy, said method comprising selecting first and second populations of cells wherein one of said first and second populations of cells is selected from a stem cell listed in Table 1 and the other of said first and second populations of cells is selected from a mature cell or stem cell such as listed in Table 1 positioning said first and second populations of cells in a fluid-filled fusing chamber and then subjecting said populations of cells to cell-fusing conditions to facilitate the fusing of at least one pair of cells.

In one embodiment, the fusing conditions comprise the addition of a chemical such as PEG. In another embodiment, an electrical current is applied to electrodes between which the cells are located having a predetermined waveform to induce the cells to fuse. After fusion, regardless of the method, the cells are cultured under conditions to permit expansion of the fused cells.

In a particularly preferred embodiment, one of the first and second populations of cells is an ES cell and the other of the first and second populations of cells is a mature cell or a stem cell from a particular part of the body.

Accordingly, another aspect of the present invention contemplates a method for generating mature cells or cells capable of differentiating into mature tissue for use in tissue replacement and/or rejuvenation therapy, said method comprising selecting first and second populations of cells wherein one of said first and second populations of cells is an ES cell and the other of said first or second populations of cells is a mature cell or a stem cell selected from the list in Table 1 and positioning said first and second cells in a fluid-filled fusing chamber and then subjecting said cells to conditions to permit fusion of at least one pair of cells and then culturing said fused cells under conditions to permit expansion of the cells.

One or both cells or populations of cells may carry a transgene, the expression of which, alters the level of differentiation or proliferation of the cells. The transgene may encode a protein (e.g. a cytokine such as LIF, G-CSF, GM-CSF, M-CSF, an interleukin, EPO, an interferon) or an RNA such as antisense RNA, sense RNA, interference (i) RNA or facilitate formulation of a complex comprising RNA. The transgene may be constitutively expressed or inducible such a developmentally regulated or regulated induced at a particular level of differentiation.

This method also includes providing a genetic sequence encoding an expression product. A gene encoding the expression product may be introduced into a cell's genome or may be introduced into the cell on a human artificial chromosome (HAC) vector such that the gene remains extrachromosomal. In such a situation, the gene is expressed by the cell from the extrachromosomal location. Vectors for introduction of genes both for recombination and for extrachromosomal maintenance are known in the art and any suitable vector may be used. Methods for introducing DNA into cells such as electroporation calcium phosphate co-precipitation and viral transduction are known in the art.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1:. 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992), murine (Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; and Baltimore, J. Virol. 54: 401-407, 1985; Miller et al., J. Virol. 62: 4337-4345, 1988) and human (Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982) origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viralvectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression may occur, for example, following direct in situ administration.

Conveniently, the first and second populations of cells are maintained in separate containers and each is selected using a macro-, micro-, nano- or pico-pipette. Consequently, the pipette selects a first cell or group of cells from a first container and then a second cell or group of cells from a second container. As described above, the first and second populations of cells are generally selected from cells listed in Table 1.

The pipette then positions the first and second cells or groups of cells generally in close proximity to each other in a fusing chamber. In the case of chemical fusion, the first and second populations of cells are generally mixed together. In the case of electrofusion, the cells are positioned and/or the electrodes are moved such that the two cells or two groups of cells are positioned substantially between the two electrodes.

Accordingly, another aspect of the present invention contemplates a method for fusing a stem cell and a mature cell or a stem cell thereof such as to generate a hybrid cell for use in developing a culture of cells for tissue replacement and/or rejuvenation therapy and/or as a source of cell-derived molecules, said method comprising selecting a first cell population from a first container and a second cell population from a second container using a pipette and placing said first and second populations of cells in a fusing chamber wherein one of said first and second populations of cells is a stem cell and the other of said first and second cells is a mature cell or a precursor form thereof and then subjecting said populations of cells to conditions to facilitate fusing of at least one pair of cells and then culturing said cells.

As above, a “population” of cells may constitute a single cell or two or more cells. Consequently, this aspect of the present invention encompasses single cell fusion and bulk cell fusion such as by chemical or electrical means or a combination of the two. The preferred form of fusion, however, is single cell electrofusion.

Accordingly, another aspect of the present invention contemplates a method for fusing a stem cell and a mature cell or a stem cell thereof such as to generate a hybrid cell for use in developing a culture of cells for tissue replacement and/or rejuvenation therapy, said method comprising selecting a first cell from a first container and a second cell from a second container using a micro-pipette and placing said first and second cells in a fusing chamber wherein one of said first and second populations of cells is a stem cell and the other of said first and second cells is a mature cell or a stem cell thereof and then applying an electrical current having a predetermined waveform to the electrodes to induce the cells to fuse and then culturing said cells.

Also as indicated above, the preferred cells are those listed in Table 1.

The pipette is designed to move one or more cells or is a micro-pipette which is ideal for capturing and transporting a single cell. In either event, the pipette is preferably coupled to a drive system adapted to move the pipette with respect to the first and second containers and the fusing chamber. The pipette comprises an actuator adapted to actuate the pipette to thereby expel or draw in fluid through a port. A controller is coupled to the drive system and the actuator moves and actuates the pipette. In operation, the controller is caused to move the pipette such that the port is adjacent to one or more cells having the desired characteristics, with the cell(s) being held in fluid suspension in the first or second container. The controller then actuates the pipette to draw in fluid through the port, thereby drawing in the cell and the surrounding fluid.

The pipette via the controller is then caused to position a second one or more cells adjacent the first cell(s) causing the controller to move the pipette such that the port is adjacent the first cell in the fusing chamber, fluid is then expelled from the micro-pipette to release the second cell(s) into the fusing chamber. If necessary, the pipette may be used to draw in fluid comprising the first and second cells or groups of cells which is then expelled back into the fusing chamber. This process may be repeated as required until the first and second cells or groups of cells are within a predetermined distance of each other.

Preferably, the pipette is a micro-pipette and is employed to manipulate single cells. This is particularly convenient for positioning cells between electrodes prior to electrofusion.

In relation to electrofusion, conveniently, the electrodes are coupled to an electrode drive system adapted to move the electrodes with respect to the fusing chamber. Generally, a controller is coupled to the electrode drive system to facilitate positioning the electrodes in the fusing chamber, and in particular relative to the first and second cells.

The electrodes are also connected to a waveform generator which applies a predetermined waveform to the electrodes leading to cell-to-cell contact, subsequent electroporation of the cells at the point of contact and, finally, fusion between the two cells leading to a hybrid fusate.

The present invention provides, therefore, a method of fusing first and second cells, the method including:
(a) selecting the first and second cells;
(b) positioning the first and second cells between two electrodes in a fluid filled fusing container, the first and second cells being held in suspension separated from each electrode; and
(c) applying a current having a predetermined waveform to the electrodes to cause the cells to fuse.

Typically, the cells are held in suspension between the electrodes.

The method typically includes generating a DEP field, the DEP field being adapted to urge the cells towards each other.

The predetermined waveform may include a current representing the DEP field. Alternatively, the method can include applying the DEP to a pair of second electrodes.

The method generally includes:
(a) applying a DEP current to the pair of second electrodes;
(b) positioning the first cell in the fusing container, the alternating field acting to attract the first cell towards one of the second pair of electrodes; and
(c) positioning the second cell in the fusing container, the alternating field acting to attract the second cell towards the first cell.

At least one of the first and second cells is generally positioned in contact with at least one of the second pair of electrodes.

The method of selecting the first and second cells typically includes using a pipette to extract:
(a) the first cell from a group of first cells held in a first container; and
(b) the second cell from a group of second cells held in a second container.

The method of positioning the first and second cells between the two electrodes usually includes:
(a) using the pipette to position the first cell in the fusing container;
(b) using the pipette to position the second cell in the fusing container, adjacent the first cell; and
(c) positioning the electrodes such that the first and second cells are located substantially between the electrodes.

The pipette is typically coupled to:
(a) a drive system adapted to move the pipette with respect to the first, second and fusing containers; and
(b) An actuator adapted to actuate the pipette to thereby expel or draw in fluid through a port.

In this case, the method usually includes using a controller coupled to the drive system and the actuator to move and actuate the pipette.

The method of selecting a cell preferably includes causing the controller to:
(a) move the pipette such that the port is adjacent a cell having predetermined characteristics, the cell being held in fluid suspension in the respective container; and
(b) actuate the pipette to draw in fluid through the port, thereby drawing in the cell and the surrounding fluid.

The method of using the pipette to position the second cell adjacent the first cell generally includes causing the controller to:
(a) move the pipette such that the port is adjacent the first cell in the fusing container;
(b) cause the pipette to expel fluid through the port, thereby expelling the second into the fluid in the fusing container;
(c) move the pipette such that the port is as close as possible to both the first and second cells;
(d) cause the pipette to draw in fluid through the port, thereby drawing in the first and second cells and the surrounding fluid;
(e) cause the pipette to expelling the first and second cells into the fluid in the fusing container; and
(f) repeat steps (c) to (e) until the first and second cells are within a predetermined distance.

The electrodes may be coupled to an electrode drive system adapted to move the electrodes with respect to the fusing containers, in which case the method typically includes using a controller coupled to the electrode drive system to position the electrodes in the fusing chamber.

The electrodes may be coupled to a signal generator, in which case the method of applying the alternating current includes causing the signal generator to apply a predetermined waveform to the electrodes.

If the first and second cells having a respective cell type, the method preferably includes using a controller coupled to the signal generator to select the current in accordance with the cell types of the first and second cells.

The first and second cells may be the same type of cell, the first and second group of cells being the same group.

In a second broad form, the present invention provides apparatus for fusing first and second cells, the apparatus including:
(a) fluid filled fusing container;
(b) at least two electrodes adapted to be positioned in the fusing container in use;
(c) a selector for:
   (i) selecting a first cell from a group of first cells held in a respective container; and
   (ii) selecting a second cell from a group of second cells held in a respective container;
   (iii) positioning the first and second cells in the fusing container, the first and second cells being held in suspension; and
(d) a signal generator coupled to the electrodes, the signal generator being adapted to cause a field having a predetermined waveform to be generated between the electrodes, thereby causing the cells to fuse.

The selector is preferably a pipette.

The apparatus generally further includes:
(a) a drive system adapted to move the pipette with respect to the first, second and fusing containers; and
(b) an actuator adapted to cause the pipette to expel or draw in fluid through a port.

The electrodes may be coupled to the fusing container.

Alternatively, the apparatus can include an electrode drive system adapted to move the electrodes with respect to the fusing containers.

The current waveform typically includes a fusion pulse, the signal generator being adapted to apply the fusion pulse to the electrodes to generate an electric field pulse thereby causing the cells to fuse.

The current waveform preferably also includes a DEP current, the signal generator being adapted to apply the DEP current to the electrodes to generate a DEP field thereby urging the cells towards each other.

The apparatus may include a pair of second electrodes, the pair of second electrodes being coupled to a second signal generator, the second signal generator being adapted to generate a DEP current, the DEP current being applied to the pair of second electrodes to generate a DEP field thereby urging the cells towards each other.

In this case, the pair of second electrodes being provided on the fusing container surface.

The apparatus also typically includes a controller adapted to control the fusing of the cells by controlling operation of at least one of:
(a) the pipette;
(b) the electrodes; and
(c) the signal generator.

The controller typically includes a processor coupled to at least one of:
(a) the drive system and the actuator, the processor being adapted to move and actuate the pipette;
(b) the electrode drive system, the processor being adapted to move the electrodes; and
(c) the signal generator, the processor being adapted to cause the signal generator to generate the field having the predetermined waveform.

The controller may include a detector adapted to detect the position of cells within the containers, in which case the processor can be responsive to the detector to move at least one of the electrodes and the pipette in response to the position of detected cells.

Alternatively, or additionally, the processing system may include an input for receiving input commands from a user.

The processor can be coupled to a store for storing waveform data representing a number of different predetermined waveforms, the processor being adapted to select one of the number of predetermined waveforms in response to the input commands received from the user.

The processor can also being adapted to move at least one of the electrodes and the pipette in response to the input commands received from the user.

Typically, the controller is adapted to cause the cells to fuse by causing the apparatus to perform the method of the first broad form of the invention.

Another aspect of the present invention provides a controller for controlling apparatus for fusing first and second cells, the apparatus including:
(a) a fluid filled fusing container;
(b) at least two electrodes;
(c) a selector;
(d) a signal generator coupled to the electrodes;
   wherein, in use, the controller is adapted to cause the cells to fuse by:
   (i) causing the selector to:
      (1) select a first cell from a group of first cells held in a respective container; and
      (2) select a second cell from a group of second cells held in a respective container; and
      (3) position the first and second cells in the fusing container between the electrodes, the first and second cells being held in suspension;
   (ii) positioning the electrodes in the fusing container; and
   (iii) causing the signal generator apply a field having a predetermined waveform to the electrodes, thereby causing the cells to fuse.

The controller can also be adapted to position the cells in the fusing container.

In this case, the controller typically includes a processor coupled to at least one of:
(a) drive system adapted to move the pipette with respect to the first, second and fusing containers;
(b) an actuator adapted to cause the pipette to expel or draw in fluid through a port;
(c) an electrode drive system adapted to move the electrodes with respect to the fusing containers; and
(d) the signal generator.

The current waveform typically includes a fusion pulse, the controller being adapted to cause the signal generator to apply the fusion pulse to the electrodes to generate an electric field pulse thereby causing the cells to fuse.

The current waveform usually includes a DEP current, the controller being adapted to cause the signal generator to apply the DEP current to the electrodes to generate a DEP field thereby urging the cells towards each other.

The apparatus can include a pair of second electrodes, the pair of second electrodes being coupled to a second signal generator, the controller being adapted to cause the second signal generator to generate a DEP current, the DEP current being applied to the pair of second electrodes to generate a DEP field thereby urging the cells towards each other.

The controller is typically adapted to operate for use with apparatus of the second broad form of the invention.

In this case, the controller is preferably adapted to cause the apparatus to perform the method of the first broad form of the invention.

In another aspect of the present invention, a computer program product is provided for controlling apparatus for fusing first and second cells, the computer program product including computer executable code which when executed by a suitable processing system causes the processing system to operate as the controller of the third broad form of the present invention.

The present invention further provides a pipette system for manipulating particles, the pipette system including:
(a) a nozzle for containing fluid in use, the nozzle including a port;
(b) an actuator coupled to the nozzle, the actuator being adapted to draw in and/or expel fluid through the port; and
(c) an electrode coupled to the nozzle adjacent the port, the electrode being adapted to cooperate with a second electrode to allow an electric field to be applied to coupled to one or more particles positioned adjacent the port.

The electrode is usually formed a conductive tube.

The electrode may be formed from a stainless steel tube having a diameter of approximately 10 mm.

The pipette system can include a drive system adapted to move the pipette system to be with respect to a fluid filled container to thereby allow particles to be positioned in or removed from fluid in the container.

The pipette system can include a signal generator coupled to the electrode for generating a predetermined electric field between the electrode and a second electrode positioned in the container.

The pipette system typically includes a controller adapted to control the drive system, the actuator and the signal generator to thereby apply an electric field to a particle by:
(a) positioning the particle in the container adjacent the second electrode using the pipette;
(b) positioning the pipette port adjacent the particle in the container; and
(c) activating the signal generator.

The controller is typically adapted to fuse cells, by:
(a) positioning a first cell in the container adjacent the second electrode using the pipette;
(b) positioning a second cell in the container adjacent the first cell using the pipette;
(c) positioning the pipette port adjacent the first and second cells, such that first and second cells are substantially between the electrodes; and
(d) activating the signal generator to cause a predetermined field sequence to be applied to the cells, thereby causing the cells to fuse.

The pipette system generally further includes:
(a) a radiation source; and
(b) a waveguide having a first end coupled to the radiation source and a second end coupled to the nozzle adjacent the port to thereby allow radiation from the radiation source to impinge on particles positioned adjacent to the port in use.

The pipette system can include a detector, the detector being adapted to detect radiation emitted by the particle.

The detector can be coupled to the first end of the waveguide, to thereby detect radiation emitted from the particle.

The radiation is typically a laser, although other sources, such as LEDs may be used.

The waveguide can be a fibre optic cable, or alternatively can being formed from the nozzle, the nozzle including a shaped portion to allow the radiation from the radiation source to enter the nozzle and pass along at least a portion of the nozzle, the radiation being emitted from the nozzle through the port.

The pipette system generally includes a controller adapted to perform at least one of:
(a) activating the actuator to thereby cause fluid to be drawn in and/or expelled through the port; and
(b) activating the radiation source, to thereby expose a particle to radiation.

The drive system can be coupled to a controller, the controller being adapted to recover particles having predetermined properties from the container by:
(a) positioning the pipette system such that the port is adjacent to a particle;
(b) activating the radiation source to thereby expose the particle to radiation;
(c) detect any radiation emitted by the particle;
(d) determine if the particle has the predetermined properties in accordance with the detected radiation; and
(e) in accordance with a successful comparison, activate the actuator to thereby draw fluid into the nozzle through the port, thereby recovering the particle.

The actuator can include:
(a) a fluid reservoir;
(b) a flexible tube coupling the nozzle to the fluid reservoir;
(c) an arm positioned so as to partially compress the tube;
(d) an actuator drive system adapted to move the arm so as to perform at least one of:
   (i) further compressing the tube to thereby expel fluid from the port; and
   (ii) decompressing the tube to thereby draw fluid in through the port.

The actuator drive system generally includes:
(a) a first actuator drive for moving the arm with respect to the tube; and
(b) a second actuator drive formed from an arm end portion, the arm end portion being in contact with the tube in use, the second actuator drive being adapted to cause the arm end portion to expand or contract.

The pipette system usually includes a controller coupled to the actuator drive system, the controller being adapted to operate the actuator drive system to thereby draw fluid in or expel fluid through the port.

The drive system can be coupled to the controller, the controller being adapted to recover particles from the fluid by:
(a) positioning the pipette system such that the port is adjacent to a particle; and
(b) activate the actuator drive system to thereby draw fluid into the nozzle through the port, thereby recovering the particle.

The tube can be formed from silicon tubing.

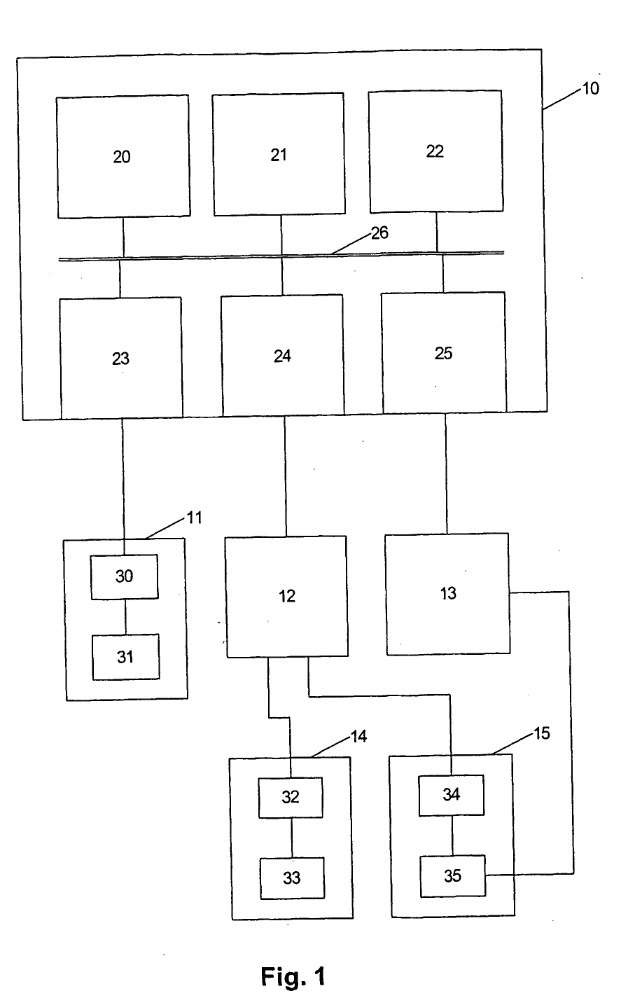

An example for apparatus suitable for implementing the present invention will now be described with reference to FIGS. 1, 2 and 3.

As shown in FIG. 1, the apparatus includes a processing system 10 coupled to an imaging system 11, a control system 12 and a signal generator 13. The control system 12 is coupled to a pipette system 14 and an electrode system 15, as shown.

The processing system 10 includes a processor 20, a memory 21, an input/output (I/O) device 22, an image interface 23, a control interface 24, and a signal interface 25, coupled together via a bus 26. The processing system may therefore be any one of a number of systems, such as a suitably programmed computer, specialized hardware, or the like. In any event, the I/O device typically includes a display, such as a computer monitor or the like, a keyboard, and one or more other input devices such as a mouse, joystick, trackball or the like.

The imaging system 11 includes a camera 30 such a CCD camera or the like which is coupled to a microscope 31. The imaging system 1 is connected to the processing system via the image interface 23.

The pipette system 14 includes a pipette shown generally at 33 that is coupled to the control system 12 via a drive system 32. In use, the control system 12 is coupled to the processor via the control interface 24, thereby allowing the drive system 32 to be used to control motion and operation of the pipette, as will be described in more detail below.

Similarly, the electrode system 15 is formed from two electrodes 35 coupled to the control system 12 via a drive system 34. Again, the control system 12 allows the drive system 34 to control the position of the electrodes, as will be described in more detail below.

In use, the system allows a user to select and move individual cells using the pipette system 14. When appropriate cells are placed next to each other, this allows an electric field to be applied to the cells using the electrodes 35 thereby causing the cells to fuse.

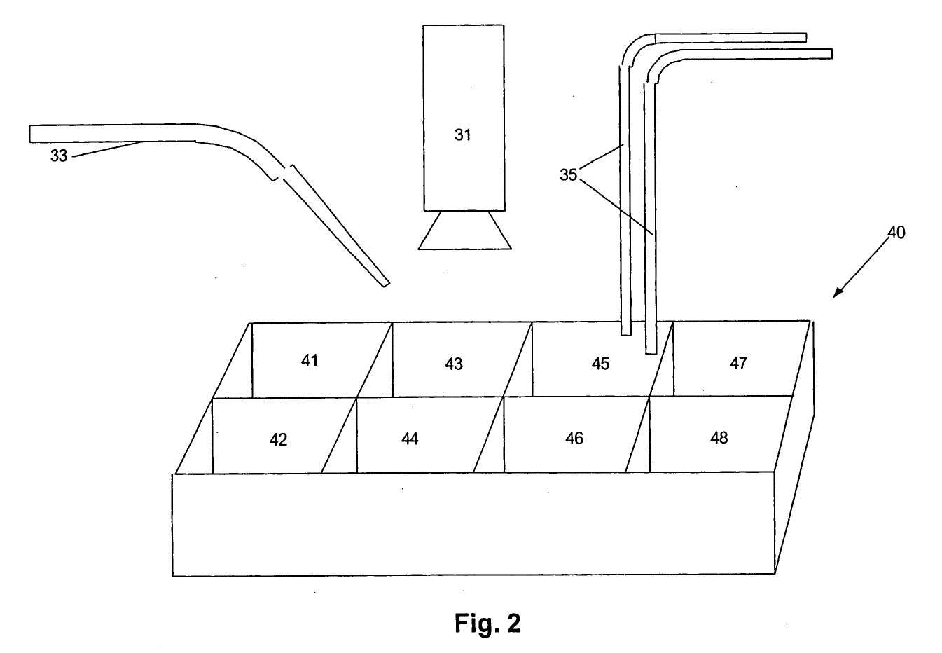

In order to achieve this, the apparatus is arranged as shown schematically in FIG. 2 such that the pipette 33 and the electrodes 35 may be moved relative to a well array shown generally at 40. This allows cells to be moved between the wells 40, 41, 42, 43, 44, 45, 46, 47, 48, as shown.

Movement of the pipette and the electrodes 35 is achieved by operation of the corresponding drive system 32, 34. Accordingly, it will be appreciated that the processing system 10 may be used to control positioning of the pipette 33 and the electrodes 35 allowing the pipette 33 and the electrodes 35 to be inserted into and positioned within a respective one of the wells 41, 42, 43, 44, 45, 46, 47, 48.

Furthermore, the microscope 31 is arranged to image selected wells 41, 42, 43, 44, 45, 46, 47, 48 such that the representation of the contents of a selected well can be displayed to the user using the I/O device 22.

In general, the processing system 10 is adapted to control the pipette 33 and the electrodes 35 in accordance with input commands received from the user via the I/O device 22. In order to achieve this, the processing system 10 must be able to perform a number of functions simultaneously, such as:
presenting an image of the well array 40 to the user on the I/O device 22;
responding to commands input via the I/O device 22 to move and, if required, actuate the pipette system 14;
responding to command inputs via the I/O device 22 to move the electrodes 35; and
responding to commands input via the I/O device 22 to apply an electrical signal to the electrodes 35.

This is achieved by having the processor 20 execute appropriate application software which is stored in the memory 21.

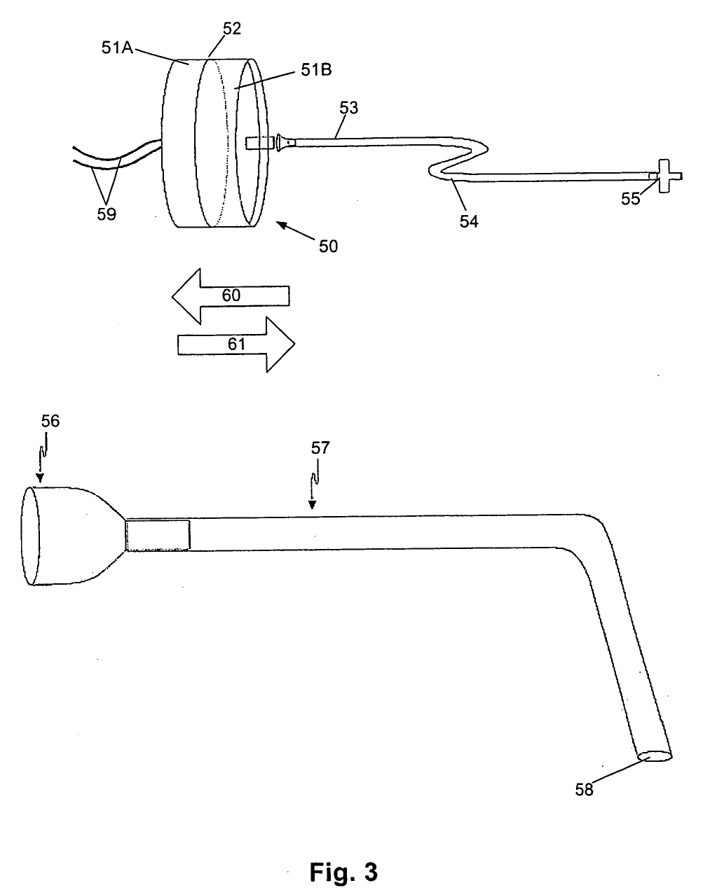

The pipette is shown in more detail in FIG. 3. As shown, the pipette 33 is formed from a housing 50 defining a chamber that is divided into two portions 51A, 51B by a piezo-electric element 52, as shown. The chamber 51B is coupled by a port 53 to a flexible tube 54. The flexible tube 54 includes a male coupling 55 that is adapted to cooperate with a female coupling 56 positioned on a shaped glass nozzle 57 having an aperture 58, as shown.

In use, the chamber 51B, the port 53, the flexible tube 54 and the glass nozzle 57 are filled with fluid, with the chamber 51A being filled with air and sealed. Applying a current to the piezo-electric element 52, via leads 59, causes the element to move, with the direction of movement depending on the polarity of the applied current.

Thus, in use, with the aperture 58 positioned in fluid in one of the wells 41, 42, 43, 44, 45, 46, 47 48, causing the piezo-electric element 52 to move in the direction of the arrow 60 will increase the volume of the chamber 51B, thereby causing fluid to be drawn through the aperture 59. Similarly, causing the piezo-electric element 52 to move in the direction of arrow 61 will decrease the volume of the chamber 51B, thereby causing fluid to be expelled through the aperture 58.

Accordingly, the pipette can be activated to draw in or expel fluid through the aperture 58 depending on the polarity of the current applied to the leads 56. Accordingly, in use, the leads 56 are coupled to either the drive system 32, or a separate activation system, to allow a suitable current to activate the pipette as required.

The manner in which the apparatus is used to fuse cells will now be described.

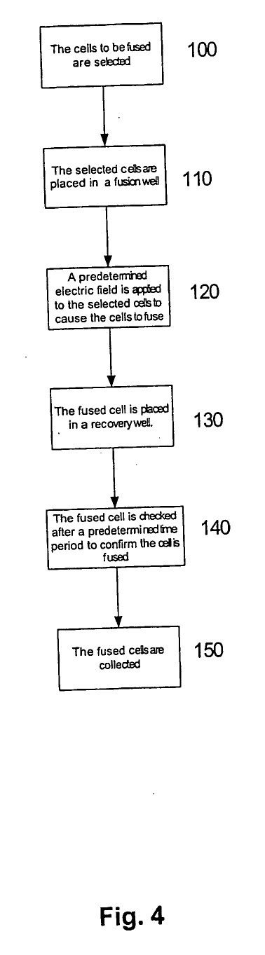

An overview of the method of fusing cells in accordance with the present invention will now be described with reference to FIG. 4.

In particular, at step 100, the user selects the cells to be fused. At step 110, the cells are placed in a fusion well.

At step 120 a predetermined electric field is applied to the selected cells to cause the cells to fuse.

Cells that are placed in an electric field will distort the field in their immediate vicinity. The field distortion is dependent on the geometry and electrical properties of the particle and that of the surrounding particles. Living cells have interior (cytoplasm) that is highly conductive, due to the accumulation of ions such as potassium (K+) ions, and a relatively high dielectric constant. The membrane surrounding has a very low conductivity and a lower dielectric constant.

Accordingly, the degree of the distortion of the field both inside and outside of the cell is a very strong function of the frequency of the applied electric field. As a result when placed in a non-uniform electric field cells will experience a force whose magnitude and direction will vary in a complicated manner with the frequency of the applied field. This effect can be exploited to selectively manipulate living cells using radio-frequency alternating electric fields created via suitable electrodes. The movement of particles in AC electric fields is referred to as “dielectropherisis” (DEP) and is independent of any net charge on the particle.

The application of the radio-frequency electric fields, typically in the region 10-10,000 kHz, exerts a positive DEP force on the two cells, urging the cells into close contact with each other. A stronger electric field is then used in order to induce electrical breakdown of each cell's membranes at their point of contact. This controlled electroporation triggers a process of cell fusion that is somewhat akin to reverse-mitosis. This in turn creates a fused hybrid cell that has a genetic make up that is a combination of the two original cells that were fused.

The fused cell is then generally placed in a recovery well at step 130 before being checked after a predetermined time period to confirm the cell has fused at step 140.

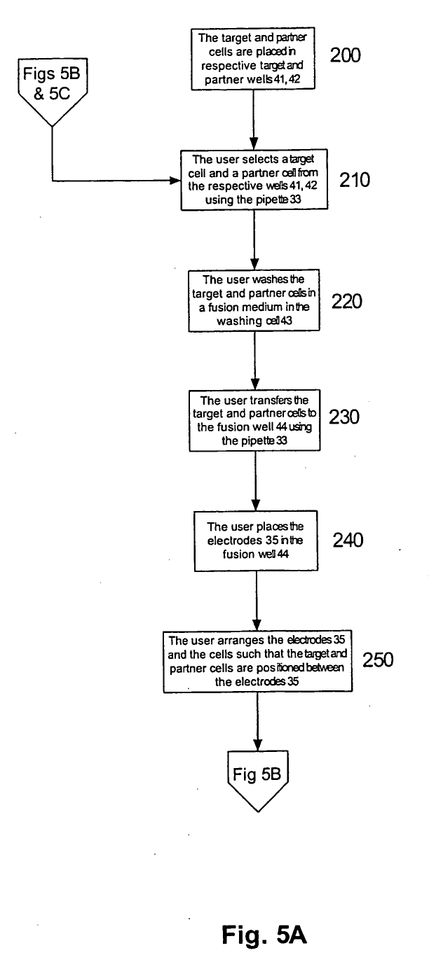

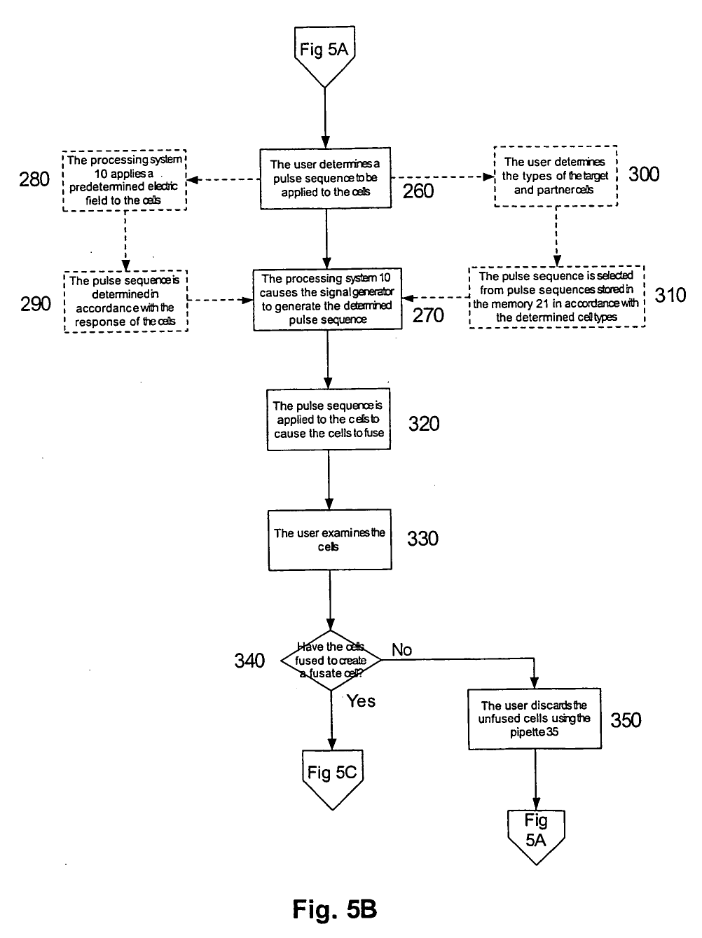

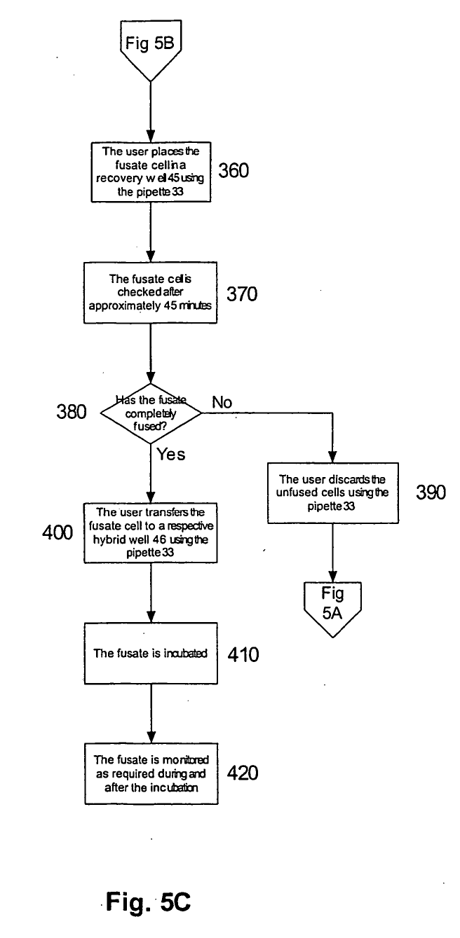

The fused cells can then be collected at 150 and used as required. detailed example of the method of using the apparatus of the present invention will now be described with reference to FIGS. 5A, 5B, 5C and 5D.

In this example, the well array 40 includes a target well 41, a partner well 42, a washing well 43, a fusion well 44, a recovery well 45 and a hybrid well 46 the purpose of which will be described in more detail below.

At step 200 the target and partner cells are placed in respective target and partner wells. This procedure will generally involve suitable preparation of the cells, which may be achieved in a number of manners. Thus, for example, this may require that the cells are recovered from sample plates and washed in appropriate enzyme solutions.

The well array would then be sterilized before appropriate fluids are inserted into the wells to be used. The target and partner cells are then placed in the target and partner wells, 41, 42 respectively, with the cells being held in suspension in respective enzyme solutions.

At step 210, the user selects a target cell from the target well 41 using the pipette 33. In order to achieve this, the user will arrange the well array 40 such that the target well 41 is imaged by the imaging system. Accordingly, the target well 41 is placed under the microscope 31 so that the camera 30 may generate an image signal and transfer this to the image interface 23. The image signal will then generally undergo some pre-processing in the image interface 23 before being transferred to the processor 20 for any subsequent further processing.

Thus, for example, the image interface 23 may be formed from an image capture card, which is used to capture images from incoming image signals. The captured image is then formatted by the processor 20 before being presented to the user using the I/O device 22.

The user adjusts the relative position of the microscope 31 and the well array until a suitable target cell is shown. The user then uses the processing system 10 to control the position of the pipette 33. In particular, this is usually achieved by having a joystick I/O device 22, with the processor 20 responding to signals from the joystick to generate commands which are transferred via the control interface 21 to the control system 12. The control system will typically be formed from a motion control addressing amplifier, which is coupled to a drive system 32, such as suitable stepper or DC servo motors.

By use of appropriate sensitivity control, this allows the position of the pipette to be controlled to high degree of accuracy. By arranging the microscope such that the pipette is shown in the image presented on the display, this allows the user to position the pipette 33 with the pipette aperture 58 adjacent the selected cell.

At this point, the user activates the pipette 33 to draw fluid in through the aperture 58. The cell and the surrounding fluid will be drawn into the pipette, allowing the target cell to be removed from the target well 41.

Sometimes, it is difficult to separate individual cells within the wells. This can be overcome by repeatedly operating the pipette to cause the pipette to repeatedly draw in and expel fluid via the pipette aperture 58. Agitation of the fluid medium and repeated movement of the cells through the pipette aperture 58 will usually allow a cell to be separated from surrounding cells.

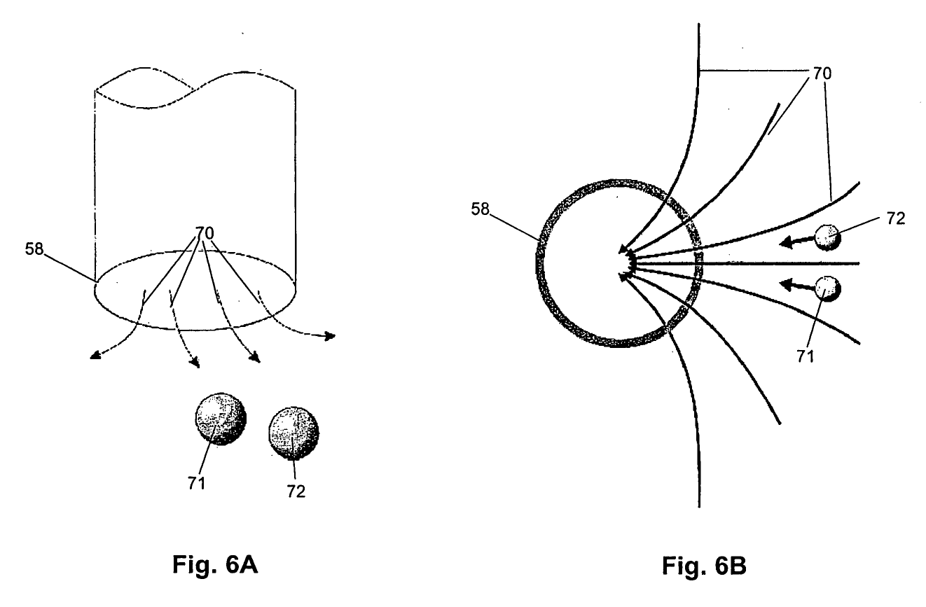

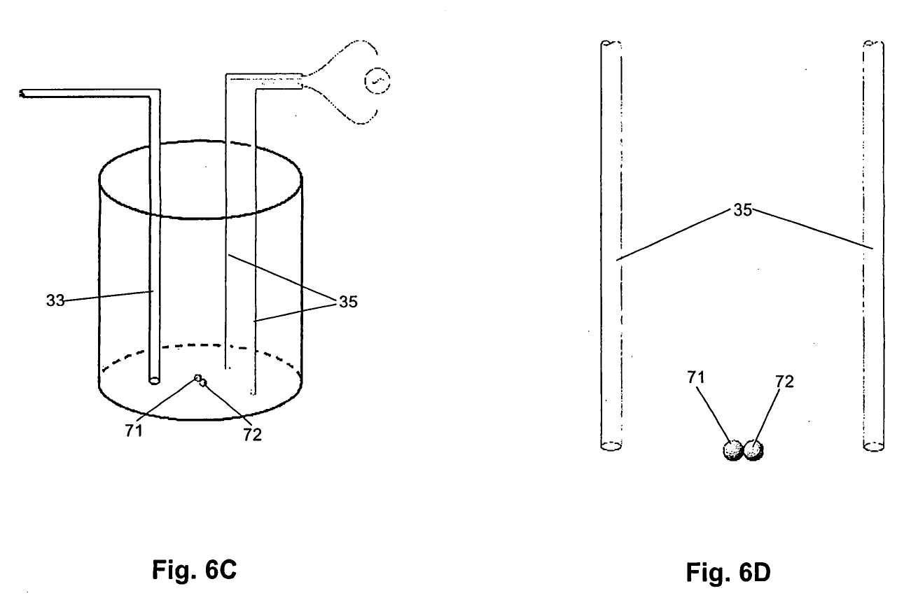

An example of this is shown in FIG. 6A, which shows the hydrodynamic stream-lines 70 as fluid is expelled from the pipette aperture 58. As shown, the hydrodynamic streamlines, which represent lines of constant force, spread out away from the pipette aperture 58. Similarly, as the cells, shown at 71, 72, are entrained in the fluid flow, this will tend to cause the cells 71, 72 to separate as they are expelled away from the pipette aperture 58.

In any event, once the user has selected the target cell at step 210, the user washes the target cell in a fusion medium in the washing well 43. In order to do this, the pipette containing the respective cell is positioned in the washing well 43, using the imaging and control system 11, 12 to move the pipette 33 as described above. Once the pipette 33 is positioned inside the washing well 43, the pipette is repeatedly activated to cause fluid to be drawn in through and expelled through the pipette aperture 58. In this way, the cell is repeatedly placed in the fusion medium in a washing well 43 and then removed. This action causes the cell to be washed.

Furthermore, when the user transfers the target and cell to the fusion well 44 at step 230, this is achieved by positioning the pipette 33 in the washing well 43 and drawing the target and cell into the pipette 33 through the pipette aperture 58. Accordingly, at this point the target cell is surrounded in fusion medium as opposed to in the medium contained in the target well 41.

The user then uses the pipette 33 to place the target cell into the fusion well 44 at step 230. Steps 210 to 230 are repeated for the partner cell, with the partner cell being placed in the fusion well 44 next to the target cell at 230.

As an alternative to performing steps 210 to 230 separately for each cell, the target and partner cells may be selected from the respective wells and then washed together in the washing cell 43 being transferred simultaneously to the fusion well 44.

As will be described in more detail below, it is preferable for the cells 71, 72 to be positioned adjacent to each other. In order to achieve this, it is preferable to first place the target or partner cell 71 in the fusion well 44 and then place the other partner or target cell 72 adjacent thereto.

In general as adding the second cell 72 will cause fluid to be transferred into the fusion well 44, this also causes movement of the first cell 71. It is then generally necessary to repeatedly activate the pipette 33 until the both cells can be drawn in to the pipette simultaneously. As shown in FIG. 6B, when the cells 71, 72 are drawn in to the pipette aperture simultaneously, the hydrodynamic lines of force 70 converge as the fluid enters the aperture 58. Accordingly, this draws the cells 71, 72 together. The cells can then be expelled from the pipette 33 with the cells being sufficiently close for the fusion process to be performed.

In any event, once the user has positioned the target and partner cells in the fusion well at 230 the user then arranges to place the electrodes 35 in the fusion cell 44 at step 240. Again, in order to achieve this, the imaging system 11 is positioned such that the I/O device 22 presents the user with an image of the fusion cell 44.

The user can then alter the position of the electrodes 35 by providing appropriate commands via the I/O device 22. Again, this is usually achieved by having a respective joystick or the like provide control signals to the processor 20. The processor then transfers appropriate command signals via the control interface 24 to the control system 12. The control system then activates the drive system 34, thereby casing the electrodes 35 to move as directed by the user.

An example of the relative positioning of the electrodes 35, the cells 71, 72 and the pipette 33 at this stage is shown in FIGS. 6C and 6D, which show a perspective and end on view of the fusion well 44 prior to fusion being performed. Thus, as shown, the cells 71, 72 are positioned close to each other substantially between the electrodes 35. At this stage the cells need not be in contact as they will in any event be urged together by the applied electrical field as will be described in more detail below.

As shown in FIG. 5B, the next step is for the user to determine the sequence of electric fields that are to be applied to the cells at step 260 before using the processing system 10 and the signal generator 13 to generate the determined pulse sequence at step 270.

The manner in which the user determines the electric field will vary depending on the particular implementation of the invention. A first example by which this may be achieved is shown in steps 280, 290. In this case, the processing system 10 applies a predetermined electric field to the partner and target cells. The response of the cells in the electric field is then used to determine the electrical parameters employed for the DEP electric field (in order to bring the cells together) The response can also be used to determine the fusion pulse sequence (including the frequency and amplitude) required to fuse any particular pair of cells. In particular, the processing system 10 will apply a field having a predetermined frequency. The frequency can then be fine adjusted until an optimum frequency is determined at which the force that attracts the cells to cells move toward each other is optimal for the required conditions. This response of the cells to the DEP electric field will occur due to the generation of electric dipoles within the cells, as described above.

The response of the cells to the electric field can be monitored either automatically by having the processor 20 execute appropriate image recognition software, or manually by the user. The processor would then select a pulse sequence from a number of pulse sequences stored in the memory 32. The pre-programed pulse sequences would be stored in a look up table (LUT), or the like, in accordance with the field applied to obtain the desired response. It will be appreciated that this information may need to be determined initially. Accordingly, each time a new lineage of target and partner cell combination is fused, the pulse sequence used to achieve this successfully will be stored in the LUT and the memory 21, together with information regarding the complete set of fusion parameters at which the desired response was observed. The processor 20 can then use the indication of the response to select a pulse sequence from the LUT.

Alternatively, the pre-programmed pulse sequences could be stored in the LUT in accordance with each particular type of target and partner cell combination. Again, this information will need to be determined initially. However, by storing the pulse sequence each time a new target and partner cell combination is fused, this allows the processor 20 to select a pulse sequence at step 310 in accordance with cell types provided by the user at step 300.

In any event, the electric pulse sequences applied to the cells to cause the cells to fuse by DEP at step 320.

At step 330, whilst the cells are still in the fusion well 44 the user examines the both morphology and the electrical behaviour of the cells to determine if they have fused to create a fusate cell. If the morphology and behaviour appear favourable to fusion then the fusate is transferred using the pipette 33 to the recovery well 45 at step 360. The initial stages of cell fusion only take a few minutes, typically under ten for most type of cells and accordingly, the user can simply view the cells on the I/O device 22 and determine from this whether the fusion process has been successful. If it is determined that the cells have not fused at step 340, the user simply discards the unfused cells with the pipette 33 at step 350, and returns to select new cells at step 210.

Once placed in the recovery well 45 the fusate cell is left for approximately 45 minutes before again being checked at step 370. During this time, the cell is held in suspension in a suitable culture medium to encourage cell growth. If it is determined that the fusate cell has not completely fused at step 380 then the user discards the unfused cells using the pipette 33 at step 390, and selects new cells at step 210.

Otherwise, the user transfers the fusate cell to a respective hybrid well 46 using the pipette 33 at step 400. The fusate cell is incubated in the hybrid well at step 410, with the cell being monitored after and during the incubation process at step 420, to determine if the fusion has been successful.

As described briefly above, different pulse sequences may be used to control the fusion of the two cells. The generation of different pulse sequences is achieved by having the processor 20 control the signal generator 13 in accordance with pre-determined pulse sequences stored in the memory 21. The pulse sequences are generally stored in data arrays and associated parameters in an LUT, as outlined above or calculated using suitable equations and data arrays at the point of fusion. The processor 20 extracts the necessary parameters and the like stored in the memory 21 and transfers this information to the signal interface 25.

In this example, the signal interface 25 is in the form of an arbitrary signal generator or the like, which uses the determined parameters to define a desired pulse sequence. The signal generator therefore generates a signal representative of the pulse sequence and transfers this to a high frequency signal amplifier, allowing the desired pulse sequence to be transferred to the electrodes 35 as required.

It will be appreciated that other forms of pulse sequence generation can also be used.

In any event, an example of different electrical pulse sequences that may be used for fusing cells will now be described. In each of these examples, the functions are defined in the temporal domain, t.

The basic pulse sequence profiles may be defined in terms of the equations:
y₁(t)=A sin(ωt)<t₁
y₂(t)=C(t)t₁<t<t₂
y₃(t)=B sin(ωt)>t₂

where:
A is a constant;
B is a constant (and may be equal to A);
C(t) is the function describing the pulse.

C(t) is typically based on one of the following functions, although it will be appreciated that this is not essential:
C₁(t)=±K
C₂(t)=Q exp(−αt)
C₃(t)=Q exp(αt)
C₄(t)=Q sin(ξt)

where:
K Q, α and ξ are constants.

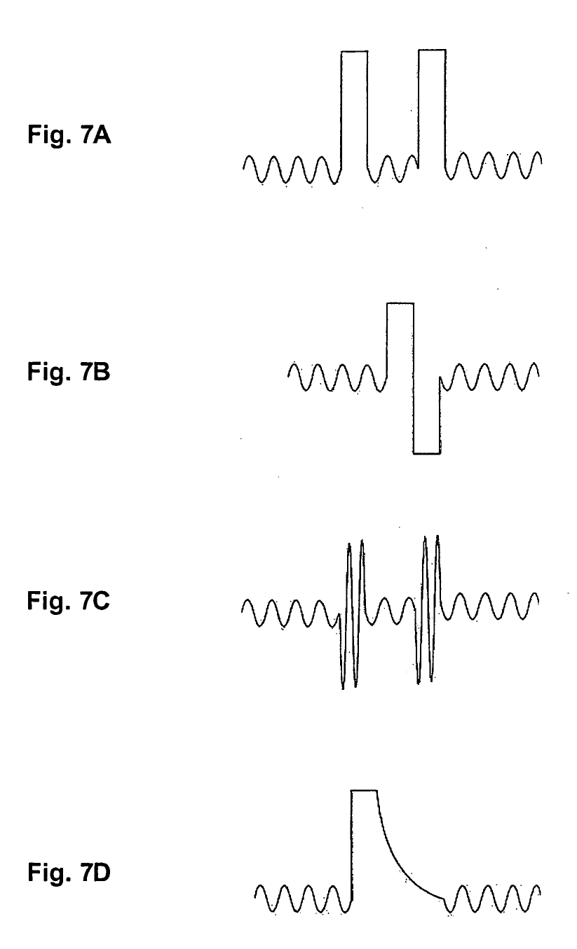

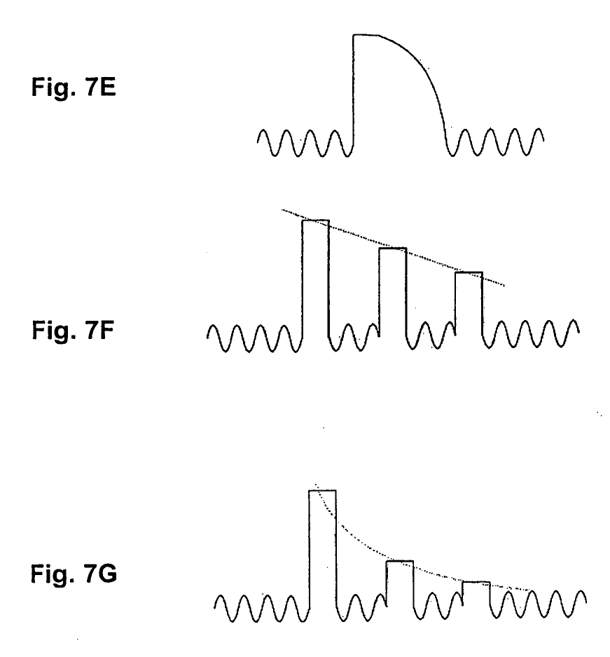

Basic pulse sequences can be combined and overlaid to create complex sequences, some examples of which are listed below and are shown in FIGS. 7A to 7G.

FIG. 7A shows a first example of a basic DC fusion pulse sequence consisting of 2 unipolar square pulses, separated by sinusoidal waves. The equations used to govern the generation of these pulse sequences are as follows:
y₁(t)=A sin(ωt)<t₁
y₂(t)=+K t₁<t<t₂
y₃(t)=B sin(ωt) t₂<t<t₃
y₄(t)=+K t₃<t<t₄
y₅(t)=A sin(ωt)>t₄

FIG. 7B shows a second example of a basic DC fusion pulse sequence consisting of a bipolar square pulse, separated by sinusoidal waves. The equations used to govern the generation of these pulse sequences are as follows:
y₁(t)=A sin(ωt)<t₁
y₂(t)=+K t₁<t<t₂
y₃(t)=K t₂<t<t₃
y₁(t)=A sin(ωt)>t₃

FIG. 7C shows a third example of a basic AC fusion pulse consisting of a sinusoidal (of differing frequency) of increased amplitude and differing frequency separated by sinusoidal waves. The equations used to govern the generation of these pulse sequences are as follows:
y₁(t)=A sin(ωt)<t₁
y₂(t)=Q sin(ξt)t₁<t<t₂
y₃(t)=B sin(ωt)t₂<t<t₃
y₄(t)=Q sin(ξt)t₃<t<t₄
y₅(t)=A sin(ωt)>t₄

FIG. 7D shows a fourth example of a basic DC and exponential pulse separated by sinusoidal waves. The equations used to govern the generation of these pulse sequences are as follows:
y₁(t)=A sin(ωt)<t₁
y₂(t)=+K t₁<t<t₂
y₃(t)=K+Q exp(−αt)t₂<t<t₃
y₄(t)=A sin(ωt)>t₃

FIG. 7E shows a fifth example of a basic DC and exponential pulse separated by sinusoidal waves. The equations used to govern the generation of these pulse sequences are as follows:
y₁(t)=A sin(ωt)<t₁
y₂(t)=+K t₁<t<t₂
y₃(t)=K−Q exp(αt)t₂<t<t₃
y₄(t)=A sin(ωt)>t₃

FIG. 7F shows a sixth example of a basic DC pulse sequence convoluted with a linear curve. The equations used to govern the generation of these pulse sequences are as follows:
y₁(t)=A sin(ωt)<t₁
y₂(t)=+K\textcircled{×}(−βt)t₁<t<t₂
y₃(t)=B sin(ωt)t₂<t<t₃
y₄(t)=+K\textcircled{×}(−βt)t₃<t<t₄
y₅(t)=A sin(ωt)>t₄

Please note that an extra DC pulse is shown in FIG. 7F for clarity.

FIG. 7G shows a seventh example of a basic DC pulse convoluted with an exponential decay curve. The equations used to govern the generation of these pulse sequences are as follows:
y₁(t)=A sin(ωt)<t₁
y₂(t)=+K\textcircled{×}(Q exp(−αt))t₁<t<t₂
y₃(t)=B sin(ωt)t₂<t<t₃
y₄(t)=+K\textcircled{×}(Q exp(−αt))t₃<t<t₄
y₅(t)=A sin(ωt)>t₄

Please note that an extra DC pulse is shown in FIG. 7G for clarity.

The subject may be any mammal such as including a human, livestock animal, laboratory tests animal or captive wild animal.

Yet another aspect of the present invention provides a composition comprising a culture of hybrid cells generated by fusing a stem cell and a mature cell or a precursor form thereof, said composition further comprising one or more pharmaceutically acceptable carriers and/or diluents.

The present invention extends to a combination of fusion techniques such as chemical and electrical fusions. Furthermore, additional selections may be made to the cells to facilitate promoting cell proximity. For example, a cell may be engineered or selected to express a ligand and another cell may be engineered or selected to express a binding portion to the ligand. Cell-cell contact is then facilitated by the ligand and its binding partner.

The fusion process and the selection of cells are conveniently used as part of a commercial operation such as in a method of doing business. For example, fusions may be done on a contract basis or entitles may specifically request certain hybrids to be generated.

Accordingly, another aspect of the present invention provides a system of cell fusion comprising:
(i) selecting two populations of cells, each population comprising one or more cells, to fuse together;
(ii) fusing the at least two cells together;
(iii) subjecting the fused cells to culture conditions to grow the fused cells;
(iv) optionally subjecting the cells to data processing means to analyze cell surface markers and/or analyzing protein or non-protein molecules produced by the cells; and
(v) providing cultures of fused cells to other entities.

This aspect of the present invention covers a method of contract research where entities either provide cell populations for fusion together and/or who seek to exploit cells which have been fused together. Such entities include inter alia patients, medical practitioners, a pharmaceutical entity or a researcher.

The fusates or hybrid cells may provide a source of new or improved molecules such as cytokines, antibodies, enzymes, proteins, heat shock proteins and ligands such a for cell receptors. The molecules may have an altered glycosylation pattern or be more stable or have greater activity or be otherwise more efficacious. The cells are said to be useful as cellular therapeutics due to their potential to produce useful molecules or large amounts of new or previously known molecules. The present invention extends to these molecules in isolated form and their production via culturing the hybrid cells.

The present invention is further described by the following non-limiting Examples.

### EXAMPLE 1

### The Selection and Fusion Chamber

A schematic of the chamber used for this particular example is shown in FIG. 4. The fusion process is carried out in a standard flat-bottomed 96 well culture plate. The wells designated for containing the cells prior to fusion are filled with a suitable growth medium and the wells designated for carrying out the fusion process are filled with a medium suitable for this purpose.

Two electrodes were mounted on a suitable drive system allowing three degrees of freedom, with one of these electrodes having a further independent three degrees of freedom. This allowed the electrodes to be moved and positioned at any location within the well.

A suitable waveform generator was connected to the electrodes that allowed a predetermined waveform to be applied across the electrodes to induce fusion between the pre-selected cells.

A pipette system suitable for the isolation and manipulation of single cells was mounted on a suitable drive system that allowed three degrees of freedom. A suitable actuator was attached to the pipette to allow fluid to be inhaled or exhaled. This system facilitates the selection and initial positioning procedure for the cells prior to the fusion process.

### EXAMPLE 2

### ES Cells

ES cells are obtained from any ethically convenient source and may be primary isolated cells or an artificially or naturally created (ES) cell line. The ES cells are dissociated into single cells and distributed into an appropriate culture vessel and medium for fusion. In some circumstances, the ES are pre-treated with proteinases or other similar enzymes.

### EXAMPLE 3

### Mature or Precursor Cells

Mature or precursor stem cells are isolated from potentially any animal tissue, but preferably human or pig tissue.

### EXAMPLE 4

### Cell Fusion

An outline of the production of fused human stem cells using the apparatus of FIG. 1 is described.

Set-Up of the Apparatus for Fusion

A 20 mL syringe (#1) was loaded with RPMI media (#2) warmed in an incubator (30 mins at 37° C.). Using the syringe a large droplet of the warmed RPMI solution was deposited into the centre of a Petrie dish (#3). This dish was then placed on the inverted microscope (Nikon TE2000) such that it was situated beneath the pipette. The pipette having first been sterilized with repeated washings of 70% v/v alcohol/water solution. The pipette was then lowered so that the tip was immersed in the droplet of RPMI. One end of a length of silicon tubing (#4) (with suitable connectors (#5)) was attached to a second syringe and the other end to the pipette. RPMI was then gently drawn into the pipette and through the tubing using the syringe. Care was taken to ensure that no air bubbles formed anywhere along the tubing or in the pipette. Using the RPMI filled syringe, fluid was injected into the nozzle of the piezo electric actuator until it was completely filled and a positive meniscus formed over the nozzle. The second syringe was then gently uncoupled from the silicon tubing. Using the first syringe filled with RPMI, the uncoupled end of the silicon tubing was topped with fluid until a positive meniscus over the mouth of the connector. The tubing was then coupled to the piezo electric nozzle 54.

Each pipette nozzle 54 is drawn from capillary tubing (120 μm inner diameter) from (#7).

The electrodes 35 were then aligned using a graticule until they were spaced ˜400-500 μm apart.

The previously prepared partner cells were then transferred to a single well in a 96 well plate (#6) and the lymphocytes were deposited in a separate well in the same plate.

The pipette was then inserted into the well containing the partner cells and a suitable partner cell selected. This (single) cell was then transferred to a fresh well containing RPMI+10% w/v fetal calf serum (FCS). The pipette was then inserted into a well containing the previously sorted B lymphocytes specific to the target antigen. A suitable B lymphocyte for fusion was then selected. Returning to the previous well the lymphocyte cell was expelled from the pipette beside the partner cell. Both cells were then visually inspected for their suitability for fusion.

Manipulation of the Cells Prior to Fusion

The pipette was then used to transfer both cells into a well containing an enzyme colution of 1% w/v pronase plus a sorbitol solution of appropriate pH and osmolarity. The cells were immersed in this medium for five minutes before being “washed” in the fusion medium (which is generally formed from a sorbitol solution of appropriate pH and osmolarity) by gently inhaling and expelling them through the pipette aperture 58 in order to allow them to acclimatize to the changed environment. Once the cells had adjusted to the change in osmolarity the pipette was then used to hydro-dynamically arrange the cells so that they were within 5-10 μm of each other. The pipette was then removed from the well.

The electrodes 35 were inserted into the well and arranged so that the previously arranged cells lay centred and co-linearly between them. Each electrode is constructed from a nickel alloy wire of 180 μm diameter manufactured by the Californian Fine Wire Company, California, USA. The configuration of the electrodes, their shape and their proximity to the cells are specifically designed so that a suitable electric field pattern can be generated in order to induce DEP between the cells.

The electrodes were connected through an amplifier to the arbitrary signal generator and a series of voltages conforming to different waveforms, previously defined by the user, were applied. The first waveform applied to the electrodes was sinusoidal and had a frequency of 500 kilohertz and an amplitude, post amplifier, of approximately 6V peak to peak. Through phenomena known as dielectropherisis, whereby neutral particles become polarized in the presence of an alternating, non-uniform, electric field, the cells experienced a force of attraction that caused them to coalesce.

The amplitude of the field was then increased to 15V peak to peak for a period of 5 seconds ensure that good membrane contact was made between the cells. In this increased field there was a slight drift of the cells towards the upper electrodes, and to counter this the stage of the microscope was adjusted relative to the electrodes to correct and retain the cells position between the electrodes.

Electrofusion of the Selected Pair of Cells to Obtain Hybrid Fusates

Once the cells were suitably arranged a field pattern conforming to the fusion pulse sequence was applied. In this instance, the fusion pulse sequence consisted of two pulse trains, each train consisting of two DC pulses, of amplitude 90V, (resulting in an electric field of approximately 180 kV) each being of 80 μs duration. The pulses were separated by a duration of 100 μs, and each train was separated by 500 milliseconds, during which in the intervening time a DEP field was applied in order to keep the cells in good contact. Post fusion pulse sequence, an increased DEP field was applied in order to maintain good contact between the cells whilst the cells fused.

Recovery of the Cells to Growth Medium

The electrodes 35 were the retracted from the fusion well, and the pipette 33 was inserted and manipulated so that the newly created fused cells were in the vicinity of the pipette aperture 58. The cells were then inhaled into the pipette and the pipette retracted from the well. In this fashion the cells were transferred to a fresh well containing hybridoma growth media (RPMI+10% w/v FCS). The newly fused cells were the only cells that were present in this media.

The above description focuses on manual use of the apparatus, in which positioning of the cells, electrodes and pipette are controlled in accordance with commands input by the user.

However, alternatively the processing system 10 can be adapted to control the apparatus automatically. In order to achieve this, the processor 20 executes image recognition applications software stored in the memory 21. This allows the processing system to use images received from the imaging system 11 to determine the position of cells within the wells 41, 42, 43, 44, 45, 46, 47 48, as well as to determine the position of the electrodes 33 and the pipette 33.

From this, it will be appreciated that the processor 20 and be programmed to perform the procedure outlined above automatically. Accordingly, the processing system will be adapted to automatically select target and partner cells in accordance with the appearance of the cell in the image. The cells will then be placed in the fusion well 44 to allow the fusion to be performed. Again, during this process the processor 20 will control the position of the cells and the electrodes.

The processor then determines the pulse sequence to be applied to the cells, and applies the pulse sequence via the electrodes 35. Once this is completed the processor 20 can monitor the cells to determine if the fusion process is successful.

Experiments have indicated that practically as few as one in seventy fused cells retain the genes needed for mitosis and of these stable cell lines a much smaller fraction go onto secrete a protein of interest. It is, therefore, desirable to have an apparatus that combined the benefits of single cell fusion along with high with a throughput of fused cells.

Examples of apparatus providing techniques for improving the throughput of the above described apparatus will now be described.

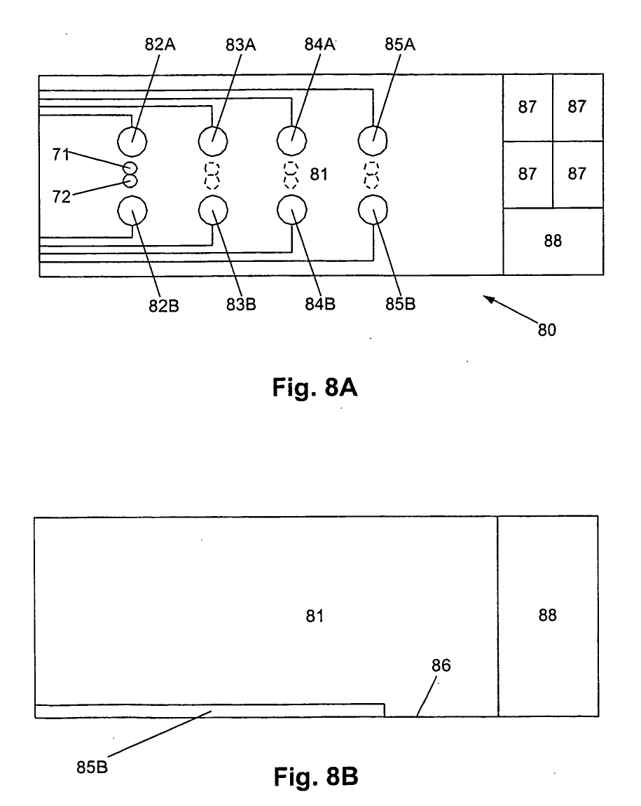

A second example of apparatus suitable for fusing cells will now be described with reference to FIGS. 8A and 8B.

In particular, the apparatus is substantially the same as the apparatus described above with respect to FIGS. 1 to 3. However, in this example, the apparatus includes a modified well array 40 having electrodes incorporated therein. Accordingly, the electrodes 35 are not required with the electrode system 15 utilizing the electrodes within the well array as will be described in more detail below.

An example of the modified well array is shown in FIG. 8A. As shown, the well array 80 includes a fusion well 81. Mounted within the fusion well 81 are a number of pairs of electrodes 82A, 82B, 83A, 83B, 84A, 84B, 85, 85B. Although only four pairs of electrodes have been shown in this Example, it will be appreciated that a greater number of electrodes may be used if an appropriately sized fusion well is provided.

The electrodes are typically formed from gold plated to a thickness of ˜2 μm onto a lower surface 86, as shown in FIG. 8B. The well array may also provided with one or more recovery wells 87, 88 as shown.

In use, the predetermined pulse sequences may be applied to the cells 71, 72 to be fused using the electrode pairs to 82, 83, 84, 85 as shown.

In use, the user will select the cells 71, 72 to be fused and position the cells between a respective pair of electrodes 82 using the pipette, as described above. Once the cells 71, 72 are positioned between the electrodes 82, the predetermined pulse sequence may be applied to the electrodes to thereby cause the cells to fuse in the manner described above.

From this it will be appreciated that four pairs of cells may be positioned in the fusion well 81 at any one time, as shown by the dotted lines. Whilst it is possible to fuse the four pairs of cells simultaneously, it is possible for the field sequence generated each pair of electrodes 82, 83, 84, 85 to interfere. Accordingly, in some cases it is preferable for each pair of cells to be fused in sequence.

In order to achieve this, the processing system 10 can be adapted to apply a first predetermined pulse sequence to the electrodes 82, followed by a second predetermined pulse sequence to the electrodes 83, etc. It will, therefore, be appreciated that different field sequences may be applied to different pairs of electrodes to allow different cells to be fused within the same recovery well.

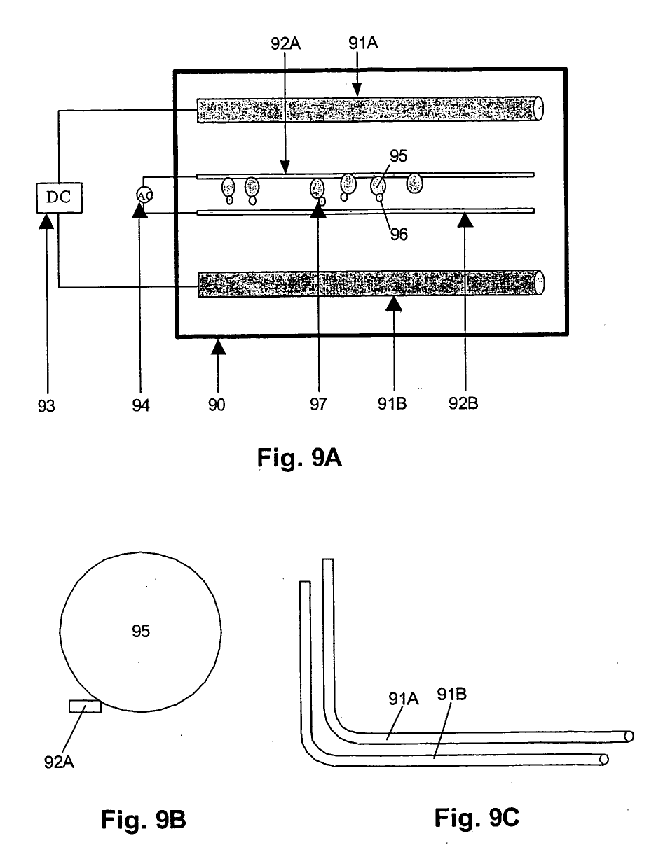

A third example of apparatus for fusing cells will now be described with reference to FIGS. 9A, 9B and 9C. In particular, FIG. 9A shows a fusion well 90 having a first pair of electrodes 91A, 91B and a second pair of electrodes 92A, 92B. In use the electrodes 91 are coupled to a first signal generator 93 with the electrodes 92A, 92B being coupled to a second signal generator 94. In this case the first and second signal generators replace the single signal generator 13 shown in FIG. 1, so that the signal generators 93, 94 are coupled to the processing system 10, via an appropriate interface 25, to allow their operation to be controlled.

In this Example, the electrodes 92A, 92B are used to generate a DEP field which is adapted to induce a dipole in cells provided at an appropriate location within the fusion well 90. This is used to attract the cells to a selected one of the electrodes 92A, 92B, thereby allowing the cells to be positioned accurately within the fusion well.

Accordingly, in use, the AC signal generator 94 will be activated to generate a DEP field. A pipette is then used to insert cells 95 into the fusion well 90, in a manner similar to that described above. In this case, the cell 95 are attracted to the electrode 92A, and will therefore align as shown. It will be appreciated that this inherent attraction reduces the accuracy with which cells must be positioned within the fusion well 90, compared to in the techniques outlined above, and will operate to retain the cells 95 in position during subsequent processing.

As shown in FIG. 9B although the cell may contact the electrode 92A, as the electrode is typically formed from a layer of gold plated onto the bottom of the fusion well 90, the point of contact between the cell 95 and the electrode 92A will typically only be very small. Thus, since these electrodes are only of the order of a micrometer high, and are only used to supply the relatively low power DEP field and not the higher power fusion pulse, as will be described below, the cells will not be damaged by the procedure, and will be easy to recover from the fusion well 90.

In any event, with a number of first cells 95 positioned in the chamber 90 a number of second cells 96 may be positioned adjacent the first cells 95. In use the dipole induced in the first cells 95 will attract the second cells 96 to form a number of cell pairs, as shown in FIG. 9A.

Once the required cells are held in position within the fusion well 90, a fusion pulse can be applied to the electrodes 91A, 91B via the first signal generator 93. This fusion pulse may consist of a simple DC current applied to the electrodes 91A, 91B, or may be formed from a more complex waveform. Similarly, the electrodes 92A, 92B are used to generate a DEP field in accordance with signals from the second signal generator.

Thus, as shown in the signals shown in FIGS. 7A-7G the overall electric field experienced by the cells consist of a generally alternating DEP field, with a superimposed fusion pulse formed from a substantially DC field. In this Example, instead of this being achieved using a single set of electrodes, the fusion pulse is produced by the first set of electrodes 91 with the DEP field being produced by the second set of electrodes 92.

In this Example described, the electrodes 91 can be provided in the cell as fixed electrodes. Alternatively, however, the electrodes may be positioned in the cell once the cells 95, 96 are in place. This has a number of advantages and in particular will avoid stray currents in the electrodes disturbing the cell placement. An example of the electrodes used in such an arrangement are shown in FIG. 9C.

This arrangement has a number of benefits.

First, allowing the first cells 95 to be placed in a DEP field generated by the electrodes 92 allows the cells to be arranged far more easily in the fusion well 90. In particular, as mentioned above, the cells 95 are held in place by the DEP field, thereby ensuring that they do not move after placement when further cells are added. This allows the cells to be placed as close as five cell diameters apart (although this is not shown in the figure for clarity) allowing a large number of cells to be aligned accurately in the fusion well 90.

Second, the second cells 96 are attracted to the first cells 95 by the generated DEP field, thereby causing the cells to naturally align to form cell pairs, as shown at 97. This vastly aids the practical speed with which cell pairs can be formed at correct locations within the fusion chamber 90. In particular, this allows a number of cell pairs to be formed in a relatively short space of time such as a couple of minutes, even using manual operation of the pipette.

Third, as the fusion pulse is provided by the first electrodes 91, the cells will not be damaged by contact with the second electrodes, thereby allowing the cells 96, 96 to be inserted into the fusion well 90 without requiring that they are positioned near to, but out of contact with the electrodes. As the cells are retained in position well away from the first electrodes 91, this allows a higher field strength to be used for the fusion pulse, which in turn increases the chances of successful cell fusion.

To further enhance this, the DEP field generated by the electrodes 92 can be momentarily increased (˜50 ms) as the fusion pulse is generated between the electrodes 91. The purpose of this is to increase the strength of the dipoles generated in the cells 95, 96, thereby urging the cells together with an increased force, to ensure good membrane to membrane contact between the cells during fusion. This helps increase the chances of successful cell fusion. Once the fusion pulse is applied the increased DEP field can be maintained for a short time after pulsing in order to further aid fusion.

Finally, a further beneficial result of this configuration is that a number of cell pairs 97 can be arranged in the fusion well 90 and exposed to substantially identical field conditions. This allows a batch of cells to be prepared having substantial identical fusate properties. This helps ensure consistency of the fusate, and allows batches of fused cells to be produced for experimental purposes.

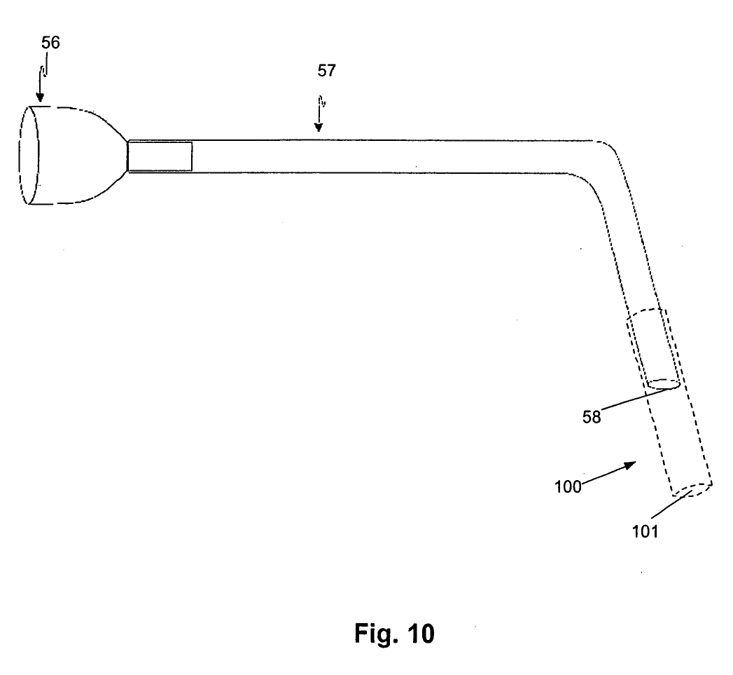

A fourth example of apparatus for fusing cells will now be described with reference to FIGS. 10 to 12.

In this Example, apparatus similar to that in FIGS. 1 to 3 is again used with one of the electrodes 35 being replaced by an electrode provided on the pipette 33. An example of the pipette is shown in FIG. 10.

As shown, the pipette is modified by the inclusion of an electrode 100 formed from a cylindrical tube 101, and which is coupled to the nozzle 57. The electrode 100 is coupled to the nozzle 57 such that the aperture 58 is contained in the tube 101 as shown.

In use, the pipette may be used substantially as described above to draw in an expel fluid through the port. This can be used to recover cells from a well allowing the cells to be placed in a fusion well, as described above.

In this example, the fusion well will additionally contain a second electrode. The second electrode may be a separate electrode similar to one of the electrodes 35 shown in FIG. 2. The cells can then be positioned between the electrode 100 and the electrode 35. The signal generator is used to apply a predetermined pulse sequence to the electrodes 100, 35, allowing the cells to be fused as described above.

Alternatively, the electrode may be provided on the underside of the fusion well, in a manner similar to that shown in FIGS. 8A and 8B.

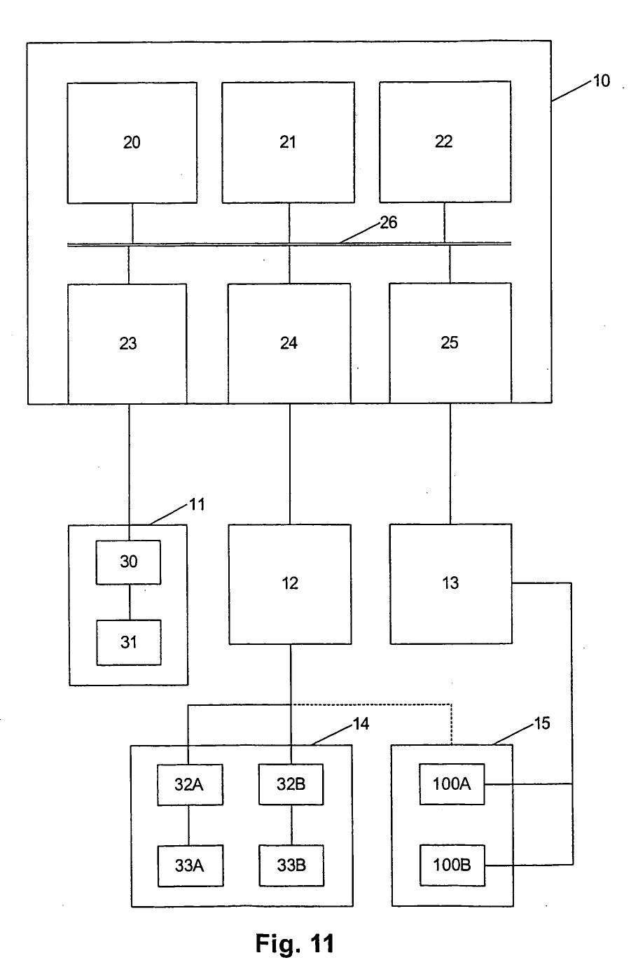

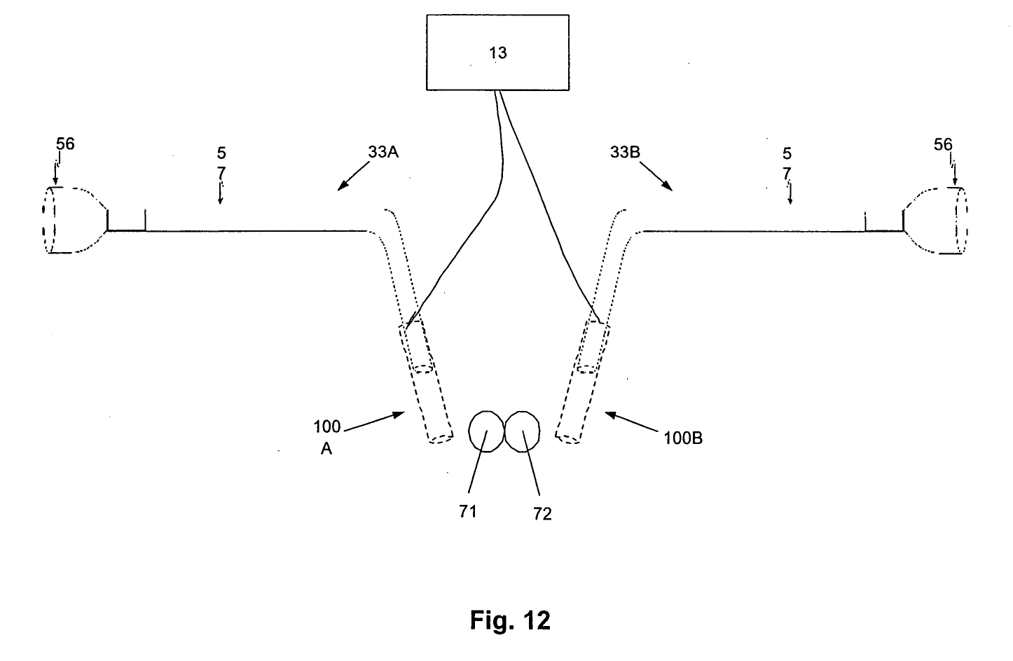

As a further option, a second pipette 33B may be provided with a respective electrode 100B. The resulting apparatus configuration is as shown in FIGS. 11 and 12, with the pipette system 14 being formed from two drive systems 32A, 32B and two pipettes 33A, 33B, as shown. Accordingly, in this example, electrodes 100A, 100B provided on the pipettes 33A, 33B, form the electrode system 15.

In any event, an electric field can be generated between the two electrodes 100A, 100B to allow cells 71, 72 to be fused in the manner described above.

It will be appreciated that the provision of a second pipette provides a number of additional advantages.

In particular, each pipette 33A, 33B is used to position respective cells 71, 72 adjacent each other by positioning the first cell 71 using the first pipette 33A, and then positioning the second cell 72 using the second pipette 33B. Once the cells are appropriately positioned, a pulse sequence can be generated between the two electrodes 90A, 90B, thereby causing the cells to fuse.

A number of additional developments can also be implemented for the pipettes. These include the provision of radiation sources such as lasers, LEDs, or the like, and appropriate detectors.

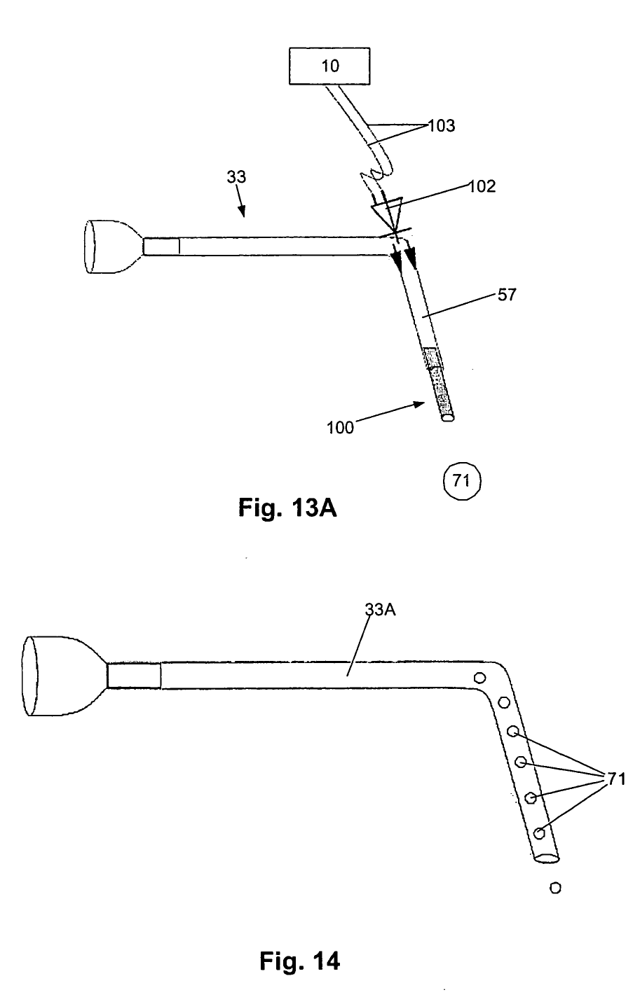

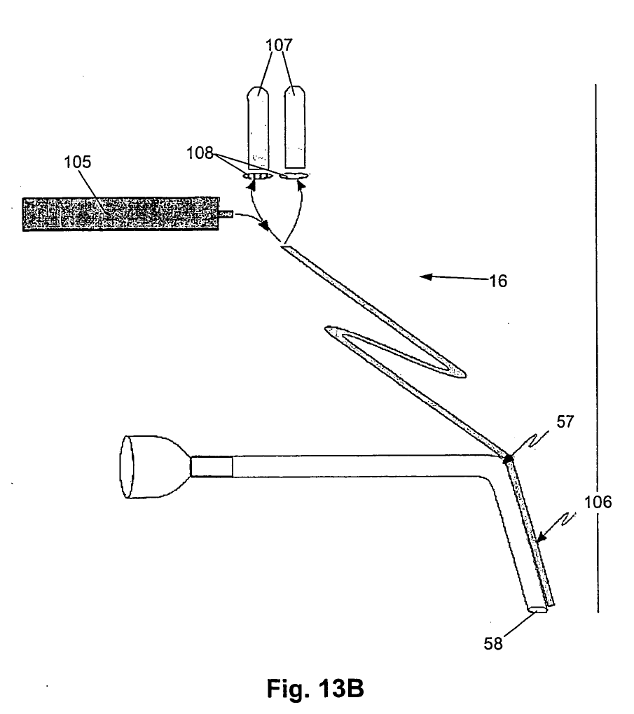

An example of this is shown in FIG. 13A. As shown, the pipette 33 includes an LED 102, arranged to direct radiation along the nozzle 57 and through the aperture 58, and electrode 100, as shown. The LED is typically coupled to the processing system 10, via leads 103, to allow the processing system to selectively activate the LED as required. This allows a cell 71 adjacent the aperture to be exposed to radiation.

This can be performed for a number of reasons. Thus, for example, this may be performed to provide simple illumination of the cells. In particular, illuminating the cells provides a increased contrast between the cell and surrounding fluid medium, thereby making it easier for the camera to resolve the cells. This in turn makes images of the cells presented to the user easier to see, as well as making automated detection of the cells easier.

In addition to this, the illumination allows cells to be labeled with fluorescent markers or the like, to allow the detectors to detect the cells having predetermined properties. In this case, visible radiation from an LED may not have sufficient power to cause the markers to fluoresce. This may be overcome achieved through the use of an LED operating at ultra-violet wavelengths. Alternatively, this may be achieved using a laser based system as shown in FIG. 13B.

In this example, a laser 105, or other radiation source such as a UV burner with suitable filters, is coupled to an optical fibre 106. The optical fibre 106 is coupled to the pipette nozzle 57, using appropriate fixing means, such as a rubber tube (not shown). The optical fibre 106 is also coupled to detectors 107, such as photo-diode tubes, via suitable filters 108.

In use, radiation emitted from the laser is used to expose cells. Any radiation subsequently reflected from, or emitted by the cells, which impinges on the fibre optic cable 106 is transferred to the detectors 107. The processing system analyzes signals from the detectors and uses these to select and remove individual cells from a group of cells held in suspension.

In addition to this, in the example of the system shown in FIG. 16, each pipette 33A, 33B could be provided with an LED 102A, 102B having a different wavelength. This allows the cells to be exposed by different wavelengths of radiation either to allow cells having different properties to be detected, for example, through the use of alternative markers, or to allow the processing system 10 or the user to determine which pipette the respective cell is near.

This also allows the processing system to use the imaging system 11 to determine from the wavelength of the radiation exposing each cell 71, 72, which pipette 33A, 33B is adjacent the cell. This also allows cells 71, 72 having different predetermined properties to be detected, by arranging for each cell to respond to a respective wavelength of radiation, for example, by the use of appropriate labels.

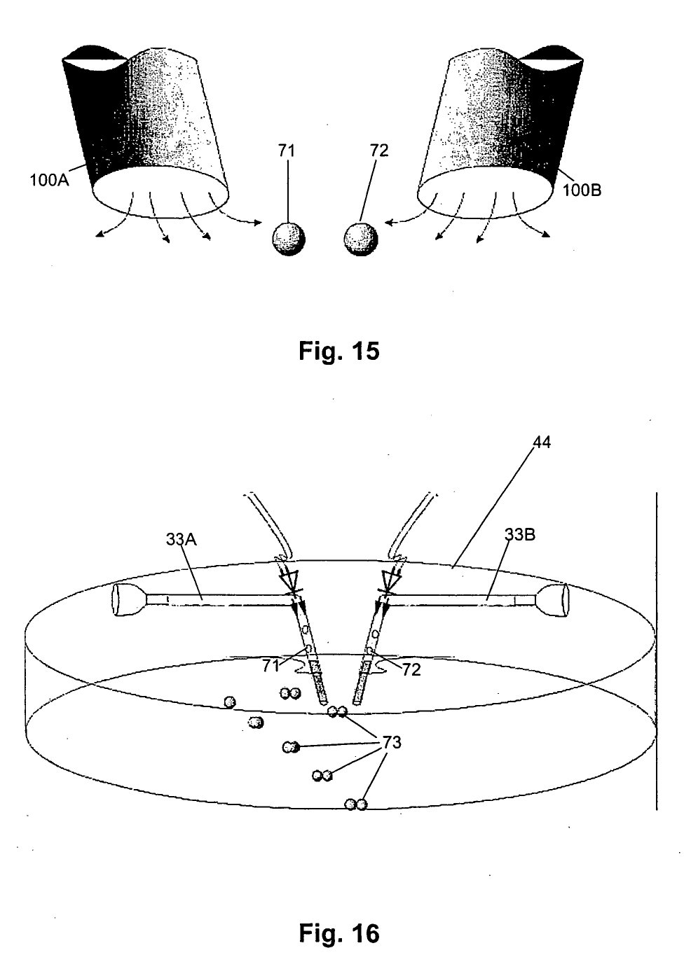

This aids in automating the system and provides for a method that allows a number of cell pairs to be rapidly fused as follows:
1. multiple cells in a source well are exposed to radiation from the LED 102A;
2. cells 71 having predetermined properties are detected by the processing system 10 and drawn into and stored in the pipette 33A, as shown in FIG. 14;
3. multiple cells in a source well are exposed to radiation from the LED 102B;
4. cells 72 having predetermined properties are detected by the processing system 10 and drawn into and stored in the pipette 33B, in a similar fashion;
5. both pipettes 33A, 33B are inserted into a fusion well 44;
6. a respective one of each cell type 71, 72, is expelled from each pipette 33A, 33B at the same time, such that hydrodynamic forces draw the cells 71, 72 together as shown in FIG. 15;
7. the processing system detects the positions of the cells using the imaging system 11 such that when the cells are expelled from the respective pipette 33A, 33B the fluid flow is truncated;
8. a DEP field is applied to draw the cells together between the electrodes, as shown for example in FIG. 12. At this point the cells are pushed together using an increased (amplitude) DEP field to aid membrane contact;
9. the signal generator 13 applies a predetermined pulse sequence to the cells 71, 72 via the electrodes 100A, 100B;
10. the cells are again pushed together using an increased (amplitude) DEP field to aid in fusion;
11. the pipettes 33A, 33B move to a new position within well;
12. steps 6-11 are repeated as many times as necessary, until a number of fusates 73 are provided as shown in FIG. 16;
13. when all cell pairs have been expelled/fused on of the pipettes travels back through the well recovering the fusates; and
14. fusates are recovered to recovery wells either as single clones or groups.

It will be appreciated that this technique can be implemented without the presence of the electrodes 100A, 100B, for example, by suitable modification of the pipette shown in FIG. 3.

There also exist techniques for labeling cells that allows them to be magnetically sorted. In this example, small metal beads are used as markers to identify cells of interest. This is achieved by ensuring that cells having desired properties can be fused to the beads and thereby extracted from a mixture of cells.

This can be achieved, for example, by coating the beads with an antibody of interest and then mixing the beads into a culture of cells. Cells that are expressing the appropriate receptor on the surface bind to the beads. The culture is then filtered through a tube, placed in an external magnetic field containing thousands of small beads that attract and hold the labeled cells, whilst allowing the unlabeled cells to be washed through and discarded. Once the external magnetic field is removed the bound cells can then be washed through the tube and isolated as desired.

It will be appreciated that this may be achieved on a smaller scale using a pipette modified to incorporate an electromagnet.

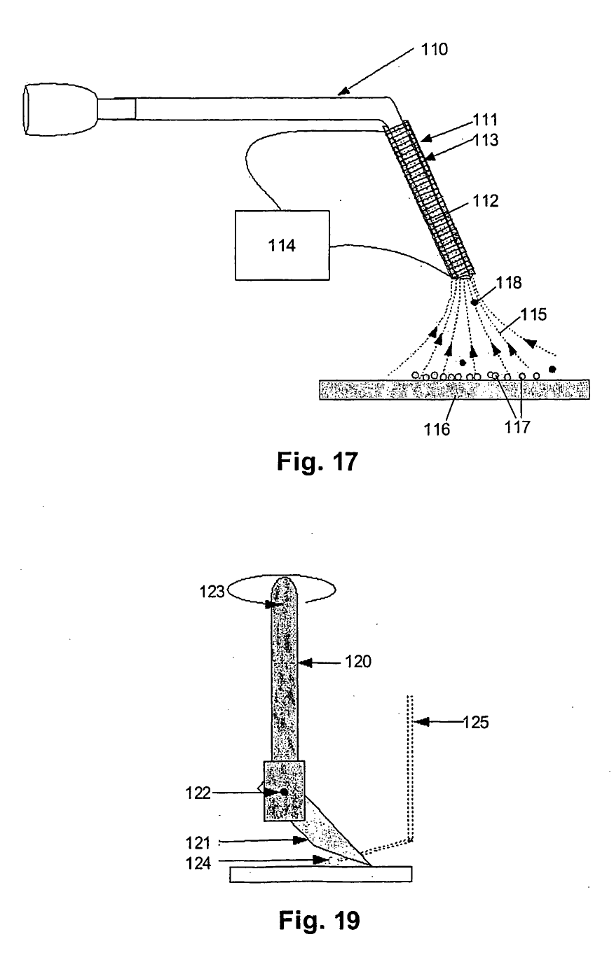

An example of a suitably modified pipette will now be described with reference to FIG. 17. In this example, the pipette shown generally at 110 includes a graphite layer 111 positioned around the pipette nozzle 112. A number of coper windings 113 are provided around a graphite core to form an electromagnet. In use, the copper windings are coupled to a DC signal generator shown generally at 114, so that the windings act as a solenoid to generate a magnetic field represented by the field lines 115.

The copper windings may be provided in a number of layers depending on the implementation and may be embedded in a layer of epoxy in order to prevent electrolysis from occurring.

The ends of the wire are connected to a variable DC signal generator and a resistor (R). Passing a current through the wire (taking account of Lenz's Law) will induce a magnetic field, the strength of which is proportional to the applied DC Voltage (V), as given by the equation.

$$B = nuI = \frac{nuV}{R}$$

where:
n=the number of turns per unit length; and
u=the permeability of free space.

In use, the pipette is positioned near a number of cells which may suspended in a fluid medium or resting on a substrate 116 as shown at 117. In this case, at least some of the cells are attached to appropriate magnetic markers, such as the beads outlined above.

In use, the metal particles, and hence the cells they are attached to, will be attracted into the magnetic field and can therefore be drawn into the pipette in the normal way. This allows cells coupled to the magnetic markers and, hence, cells having certain properties to be selected.

It will be appreciated that cells with a higher density of receptors (a higher number of magnetic markers) should have a larger force exerted on them than cells with less receptors for the same magnetic field strength. Therefore, as the DC voltage is increased, a larger number of cells should be drawn into the magnetic fields influence. This field gradient can allow for a further sorting criteria.

In order to ensure no wanted cells have been collected, it is possible to flush out the pipette by expelling fluid from the nozzle. In this case, any cells not bound magnetic markers will be expelled from the pipette together with the fluid, whilst the cells bound to markers will be held in place by the action of the magnetic field. In this case, when the selected cells are to be expelled, the magnetic field can be deactivated allowing the cells and attached markers to be expelled in the normal way.

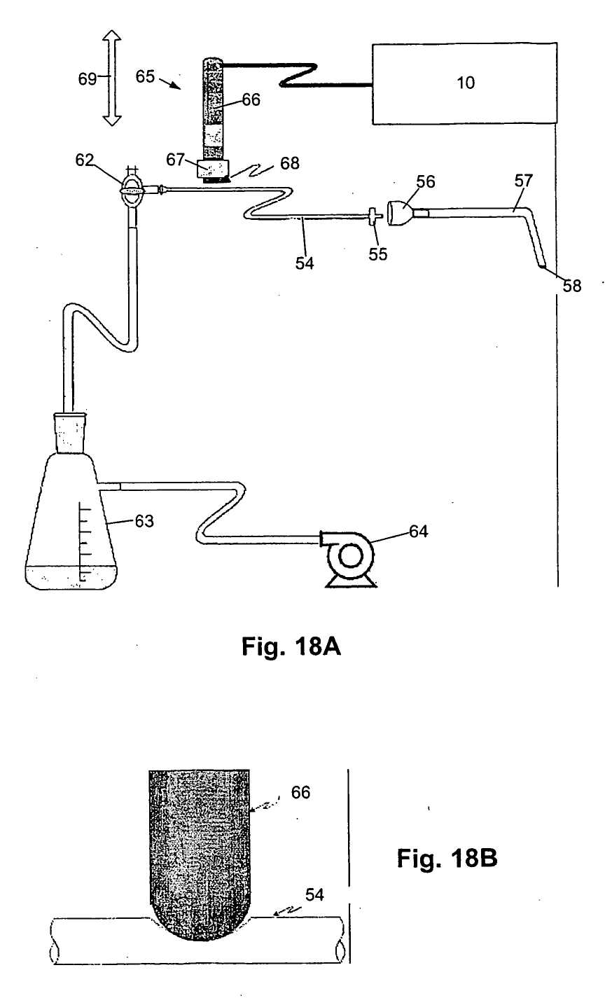

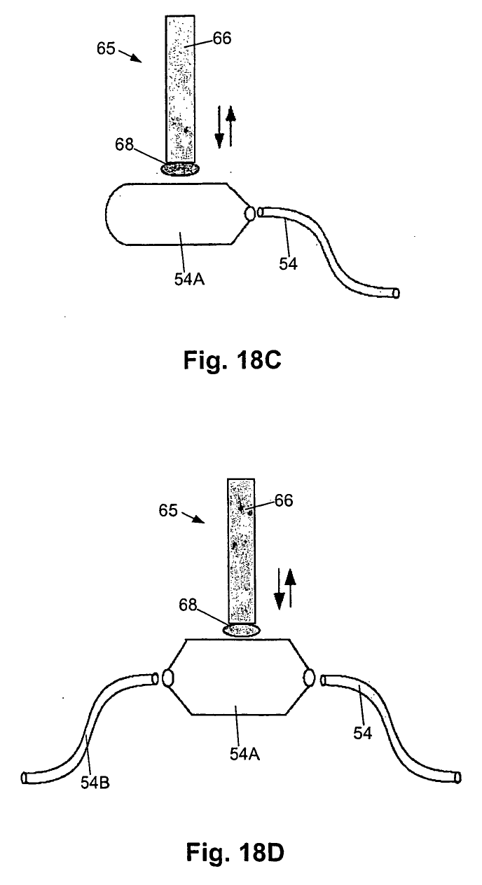

A further development is for an alternative form of actuator to be used. An example of this form of actuator is shown in FIGS. 18A, 18B.

As shown, in this example, the tube 54 is connected via a stopcock 62 and a reservoir 63 to a pump 64. An actuator 65 is positioned adjacent the flexible tube 54, to allow the tube to be clamped as shown in FIG. 18B.

It will be appreciated from this that any form of actuator, such as a solenoid, may be used. However, in this example, the actuator is formed from a threaded screw drive 66, coupled a DC or stepper motor 67, which forms part of the drive system 32. In use, this allows the actuator to be moved in the direction of the arrow 69, an amount of ±5 mm.

The actuator tip can have a piezo electric stack 68 coupled thereto, to allow fine control (displacement of ±20 μm) of the end of the actuator. Again, the piezo stack forms part of the drive system 32.

In use, the pipette is loaded with a suitable fluid medium by placing the aperture 58 into a container that has sufficient fluid to fill the system. The pump or other such means of drawing fluid through the system is activated and fluid is drawn through the pipette nozzle 57. When the system is loaded and there are no air bubbles present in the tubing, the stopcock 62 is closed to prevent further fluid flow, and the pump 64 turned off.

Whilst the aperture 58 is still immersed in the fluid medium, the actuator 65 is adjusted such that the silicon tubing 54 is compressed to about half its diameter, as shown in FIG. 2B. Thus, in use, with the port 41 positioned in fluid in a well causing the actuator 65 to move in the direction of the arrow 69 compresses or releases the tubing 54 which, in turn, either expels or draws in fluid through the port 41. This allows cells to be recovered from a well as described above with respect to the pipette of FIG. 3.

Variation on this are shown in FIGS. 18C and 18D. In these examples, the actuator 65 is positioned adjacent a bladder 54A provided in the flexible tube 54. In this case, the bladder has a larger cross sectional area than the tube and will, therefore, contain a greater volume of fluid per unit length compared to the tubing 54. This has two main benefits. In particular, the larger cross sectional area provides for a greater range of movement of the actuator. This coupled with the increased fluid volume in the bladder allows for a greater amount of fluid to be displaced when compared to the action of the actuator on the tube 54.

As a result this provides greater control over the amount of fluid expelled or drawn in through the aperture 58, allowing for greater accuracy in retrieving individual cells using the pipette.

In this instance, it will be appreciated that by providing sufficient liquid in the bladder, it is not necessary to provide the stopcock 62, the reservoir 63 or the pump 64 as shown in FIG. 18C. In particular, the bladder and pipette can be filled, with an amount of fluid being expelled from the bladder before the bladder is positioned so as to cooperate with the actuator, thereby allowing the actuator position to be adjusted to allow fluid to be drawn in or expelled through the aperture 58.

Alternatively, the bladder can be connected to a stopcock 62, reservoir 63 and pump 64, by a tube 54B, as shown in FIG. 18D.

Accordingly, the system described above allows individual cells to be easily fused. As the cells are manipulated using the pipette as shown in FIG. 3, this makes the cell manipulation far easier than in the prior art. This, therefore, helps increase the speed and ease with which fusion of individual cells can be performed. Furthermore, the electrodes need never touch the cells, thereby helping reduce or prevent cell damage prior to or during the fusion process.

In addition to this, the apparatus as a whole is generally less complicated, thereby helping reduce the cost, as well as easing use of the apparatus to perform cell fusion. As a result, fusion using the system described above can generally be achieved more rapidly and cheaper than in the prior art.

A further development that can be utilized within the examples described above is for a cutting tool to be provided to allow cells to be cut, as well as to allow cells that have adhered to the well surface or electrodes to be released. An example of a suitable cutting tool is shown in FIG. 19. As shown, the cutting tool includes a support post 120 having a blade 121 pivotally mounted thereto by a hinge 122 or other appropriate connection.

In use, the post is coupled to a micro manipulator (not shown), to allow the post to be positioned within the respective well. The post can be rotated as shown by the arrow 123, allowing the blade to be positioned above a cell to be cut. If the cell is a free cell 124, the cell will generally be held in place using a pipette, or other suitable manipulator, as shown at 125.

Once positioned, the post is lowered such that the tip of the blade “bites” into the soft plastic of the bottom of the plastic plate. Further lowering of the post will cause the blade to pivot around the hinge 122 and “guillotine” through object, such as the cell, placed in its path. Motion is stopped when the blade has cut through the object of interest and is completely parallel with bottom of plate.

It will be appreciated that the functionality of the different examples described above may be combined in any one of a number of arrangements. This allows for example cells to be selected automatically in accordance with magnetic or radiation sensitive markers. The cells can then be arranged in a fusion well, and fused, with the fusate being automatically retrieved and positioned in a recovery well.

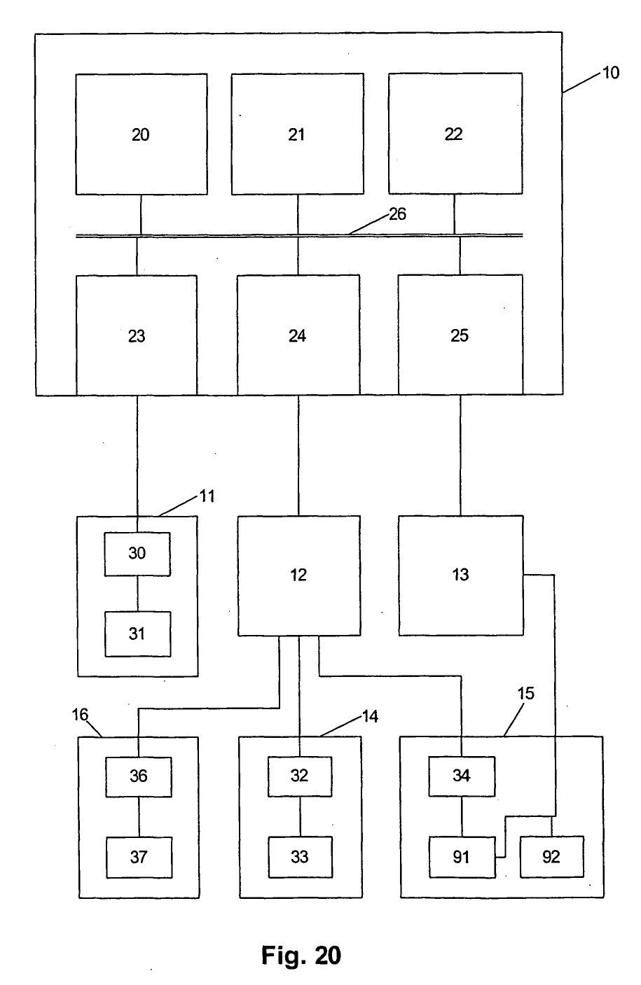

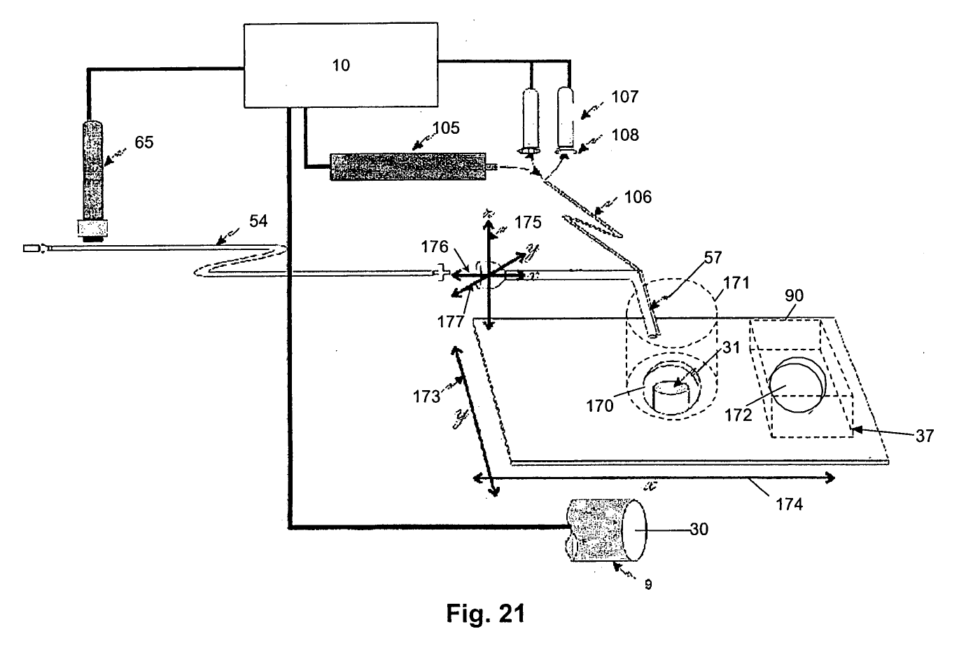

A specific example of apparatus for performing automatic cell selection and fusion will now be described with reference to FIGS. 20 and 21. As shown in FIG. 20, the control system 12 is further coupled to a stage system 16, including a drive system 36 coupled to a stage 37, with the processing system 10 being coupled to a stimulation system 17. The stimulation system 17 is used to stimulate cells, to allow cells having predetermined properties to be recovered from a group of cells held in suspension in a selection well.

In order to achieve selection the cells are labeled with markers, which are adapted to adhere and or permeate only the cells having the required predetermined properties. The stimulation system 17 stimulates the marker cells and thereby identify the cells having the predetermined properties. It will be appreciated that the stimulation system 17 may be a radiation based system, similar to that described with respect to FIG. 13, or a magnetic based system similar to that described with respect to FIG. 17. The following example will focus on the use of a radiative based approach.

The arrangement of the apparatus is shown in more detail in FIG. 21.

As shown, the stage 37 includes an aperture 170, having the microscope 31 mounted therein. From this, it will be appreciated that the microscope 31 is typically an inverted microscope.

In use the stage 37 is adapted to receive a selection well 171 containing the cells to be recovered. The stage will also receive a fusion well 90, positioned over an aperture 172. In use, the selection well 171 is positioned on top of the aperture 170, to allow the camera 30 to obtain an image of the inside of the selection well 171, via the microscope 31. In use, the processing system 10 is adapted to control the drive system 36, to cause the stage 37 to be move in the directions shown by the arrows 173, 174.

This allows a representation of the contents of a selected well can be captured by the processing system 10 using the image interface 23, which is typically a frame grabber or the like. Images may then be used by the processing system to control the drive systems 32, 35 and 36 and the stimulation system 17. Additionally or alternatively, images may be displayed to a user using the I/O device 22.

The pipette is positioned adjacent the stage 37 as shown, to allow the nozzle 57 to be inserted into the well 171. The pipette 33 is coupled to the drive system 32, to allow the pipette to moved with respect to the well, as shown by the arrows 175, 176, 177. Accordingly, the drive system 12 typically includes a micromanipulator system having three independently controlled axis with resolution tolerances and repeatabilities of <5 μm. This system is controlled by dedicated software executed by the processor 20.

In any event, the cells having the predetermined properties are identified by exposing the cells to radiation using the radiation source 105 coupled to the nozzle 57 via the fibre optic cable 106. This allows the detectors 107 to receive radiation emitted by the cells through the fibre optic cable 106 and filters 108, to thereby determine cells having desired properties.

It will be appreciated that in the event that the detection of particles is performed magnetically, this may be achieved as described above with respect to FIG. 17.

The processing system 10 can then control the pipette system 14 to remove cells from the selection well 171 and place these in the fusion well 90, as described above. During this process a DEP field will be applied to the electrodes 92 to ensure the cells are positioned as required. In addition to this, the stage 37 is moved, to allow the camera 30 to image the fusion well 90 through the aperture 172.

Fusion will then be performed substantially as described above, with the fused cells being removed as required.

Accordingly, the above system describes apparatus suitable for manipulating and fusing cells, and in particular for single cell, mini-bulk or macro-bulk cell fusion. In this regard, the term cells is intended to cover any cells, vectors, particles, molecules, liposomes, and other such vesicles.

This allows the techniques to be used for generating tissue or cells useful for tissue replacement and/or tissue rejuvenation therapy or a range of organs or tissue areas of the body. The resulting tissue or cells may also secrete or generate a range of cytokines, enzymes, hormones and the like which have improved or more efficacious properties relative to analogous molecules produced from non-fused cells.

In this case, the cells are selected to have desirable properties, such that the generated fusate has properties applicable for a specific purpose.

A suitable list of stem and mature cells and their application for use in transplant and rejuvenation therapy is shown in Table 1. All such stem and mature cells are contemplated and are encompassed by the present invention. As indicated in Table 1, a mature cell may be derived from any human or mammalian or non-mammalian animal or avian species such as from the brain, epidermis, skin, pancreas, kidney, liver, breast, lung, muscle, heart, eye, bone, spleen or the immune system. Cells of the immune system include CD4+ T-cells, CD8+ T-cells, NK cells, monocytes, macrophages, dendritic cells and B-bells. It should be noted that the present invention contemplates the fusion of stem cells and mature cells from any source such as a mammal (including human), non-mammalian animal and avian species. Examples of non-human mammals include livestock animals (e.g. sheep, pigs, cows, horses, donkeys, goats), companion animals (e.g. cats, dogs), laboratory test animals (e.g. mice, rats, rabbits, guinea pigs, hamsters) and captured wild animals. A non-mammalian animal includes a reptile, amphibian, insect, arthropod and arachnids. Avian species include poultry, birds (e.g. ducks, emus, ostriches) and aviary birds.

In terms of using the cells for tissue replacement therapy or augmentation therapy, at least one population of cells may come from the subject to be treated or from a histocompatibility matched subject (i.e. an HLA-matched subject). Furthermore, at birth, subjects may store cells or tissue for the use of the subject (or other suitable subject) later in life. Such tissue would include placenta tissue, umbilical chord tissue, foreskin, blood or other uteric tissue associated with a fetus.

Accordingly, while the above description has focused on cell fusion, it will be appreciated that the techniques may generally be applied to any cells, vectors, particles, molecules, liposomes, and other such vesicles.

### EXAMPLE 5

### Chemical and Electrical Bulk Fusion

Cells are collected in a tissue culture or similar plastic vessel and placed in the electrofusion chamber or other fusion chamber for use in a chemical fusion process. If the cell fusion process occurs by utilizing electrofusion, then the cells are generally micro-manipulated to be placed in a physically close proximity to each other in a medium. Cell-cell fusion is generally conducted between two single cells of the types described herein although fusion may also be done in a bulk manner with potentially tens, hundreds, thousands or millions of cells or more.

FIG. 6 shows a process of bulk chemical (A) or electrical (B) cell fusion. FIG. 7 is a diagrammatic representation showing single cell electrofusion.

Upon fusion, in the case of cells fused in pairs, the resulting fusion consists of the nuclei of the two original cells encased in the fused lipid bi-layer from the two original cells (FIG. 8). The nuclei of the cells will fuse over time resulting in a cell with an abnormal number of chromosomes which might be quadraploid or contain less or greater number of chromosomes. This cell has the ability to divide and proliferate under the appropriate culture and nutrient conditions.

### EXAMPLE 6

### Fusion Between ES Cells and Adult Neural Stem Cells

Using the methods described herein, a single cell fusion is conducted between an ES cell and an adult neural stem cell.

### EXAMPLE 7

### Fusion Between ES Cells and Bone-Marrow Derived Mesencityinal Stem Cells

Using the methods described herein, a single cell fusion is conducted between an ES cell and a human mesenchymal stem cell. The mesenchymal stem cell has a transgene encoding a myosin heavy chain gene product. The two cell types are fused together using single cell electrofusion and recovered in a suitable culture medium, and allowed to divide over ensuing days. The fusates are expanded to a population of several million, then finally treated with the addition of a chemical agent to induce differentiation to a “beating” cardiomyocyte cell type. The resulting cells are potentially useful for therapy of damaged heart tissue following heart attack.

### EXAMPLE 8

### Fusion Between ES Cells and Human Foreskin Fibroblast Cells

Using the methods described herein, a single cell fusion is conducted between an ES cell and a human foreskin fibroblast cell.

### EXAMPLE 9

### Fusion Between ES Cells and CD34+ Stem Cells

Using the methods described herein, a single cell fusion is conducted between an ES cell and a CD34+ stem cell.

### EXAMPLE 10

### Fusion Between Neural Stem Cells and Astrocyte Cells

Using the methods described herein, a single cell fusion is conducted between a neural stem cell and an astrocyte cell.

### EXAMPLE 11

### Fusion Between Neural Stem Cells and Human Lung Fibroblasts

Using the methods described herein, a single cell fusion is conducted between a neural stem cell and a human lung fibroblast.

### EXAMPLE 12

### Autofusion of ES Cells

R1ES cells were cultured in ES medium (with 10% v/v serum). The fiusion mediuim used was a suitable fusion medium.

R1ES cells along with feeder cells were placed into one well of a 96 well plate. The well contained ES culture medium. Cells for fusion were selected by eye according to their morphology. Pairs of cells were brought into a well containing SCB and manipulated into contact by hydrodynamic focusing and dielectrophoresis. Cell fusion was induced by the application of brief D.C. pulses as indicated below. After the cells were pulsed they were removed from the fusion well and placed into a fresh well containing ES culture medium.

### EXAMPLE 13

### Cell Selection Method

The following is a method for recovering fused cells following mini-bulk or macro-bulk fusion of cells by either chemical, electrical or other means.

One of the issues for the recovery of fused cells following a bulk fusion is that there often needs to be a selection process to sort out fused cells from unfused cells. This is because only a tiny proportion of cells in a bulk fusion will actually fuse (perhaps as little as 0.001%), leaving a mixture of largely unfused cells. One of the most commonly used selection processes in this context is HAT selection. The problem with this method is that fusion is limited to partner cells that are HAT sensitive. HAT sensitive cell-lines take a large investment in time and resources to generate. This is a major limitation with respect to stem cells and embryonic stem cells, as these are isolated cells, or are indeed very difficult to culture. Certainly, manipulation of HAT sensitivity properties in an ES cell-line would appear to be an incredibly difficult objective.

Another approach might be to sort out the cells based on surface markers. This is a well-established technology that generally relies on antibodies conjugated to molecules that fluoresce upon excitation with a laser (antibody-based FACS sorting technology). Under this regime, one would label the two different cell populations that one planned to fuse with two different surface marker reagents respectively. The two reagents might fluoresce red and green respectively, and thus any cell fusion instances (fusates) could be readily sorted and separated on the basis that they showed both red and green detection rather than only one of the two like fused cells. This has the advantage of sorting for specific cell types in a mixture, defined by their surface expression. There are, however, several problems with this approach. Firstly, the surface markers may be down-regulated following fusion of two different cell types, thus potentially removing the ability to retrieve rare fusates from the large mixture of unfused cells. Secondly, a specific surface marker will be needed for every cell type you wish to fuse. A specific surface marker may not have been developed for many rare cell types, including stem cells. Thirdly, if you plan to fuse a complex mixture of cells, for example, a preparation of whole splenocytes, then by using a single surface marker, you may fail to recover certain fusates of cells that do not express that surface marker.

What is ideally needed is a generic, non-specific stain that can be used for all types of cells irrespective of the surface markers they express. Such stains are now available. CFSE is a reagent that permeates cells, and then once inside, chemically converts to a membrane impermeable form. Thus it is trapped inside the cell as an intracellular dye. CFSE fluoresces green under laser excitation. CFSE has been used extensively by Australian immunologists for many years to track the cell divisions of primary B-cells. The dye is a little toxic to cells, but when used optimally will allow the cells to divide for 10 cycles or more, and is gradually diluted through each cycle, suggesting that the dye can be safely used to recover viable cells. Other similar reagents in other color regions are available, thus making it possible to FACS sort fusates in an analogous approach to that described above. It is noted that researchers have previously used toxic intracellular dyes to monitor the efficiency of macro-bulk electrofusion of mixtures of two cell types. However, what is not recorded (or presumably invented) is the application of using slightly toxic intracellular dyes under optimal conditions to recover fused cells for ultimate culture and expansion following dilution and ridding of the remnants of the dyes. This method would be conducted in combination with fluorescence microscopy and FACS sorting.

It is proposed that this two color-based method be used in combination with stem cell mini-bulk and macro-bulk fusion to produce a powerful method of separating a small population of fused cells (fusates) from a large number of unfused cells in a complex mixture.

### EXAMPLE 14

### Fused Cells as a Therapeutic Treatment for Insulin-Dependent Diabetes Mellitus

Using the methods described herein, fused cells could be-used as a therapeutic treatment for insulin-dependent diabetes mellitus. In this approach, ES cells could be fused with pancreatic β-cells or duct cells that may or may not be autologous with the subject. The hybrid cells could then be propagated in vitro prior to being transplanted into an affected patient's pancreas to reinstate the normal function of the affected organ.

### EXAMPLE 15

### Fused Cells as a Therapeutic Treatment for Parkinson's Disease

Using the methods described herein, fused cells could be used as a therapeutic treatment for Parkinson' disease. In this approach, ES cells could be fused with dopamine-secreting cells, preferably neurons, that may or may not be autologous with the subject. The hybrid cells could then be propagated in vitro prior to being transplanted into an affected patient's brain to reinstate the normal function of the affected organ.

### EXAMPLE 16

### Fused Cells as a Therapeutic Treatment for Nervous System Trauma

Using the methods described herein, fused cells could be used as a therapeutic treatment for nervous system trauma. In this approach, cells secreating a therapeutic agent could be fused with astrocytes or oligodendrocytes that may or may not be autologous with the subject. The hybrid cells could then be propagated in vitro prior to being transplanted into an affected patient's nervous system to promote regeneration in affected areas.

### EXAMPLE 17

### Fused Cells as a Source of Cell-Derived Therapeutic Agents

Using the methods described herein, fused cells could be used as a source of cell-derived therapeutic agents such as cytokines, enzymes and hormones that have improved or more efficacious properties relative to analogous molecules produced from non-fused cells. In this approach, cells secreting a therapeutic agent could be fused with stem cells. The hybrid cells could then be maintained and propagated in vitro as a source of therapeutic agents that can be isolated and purified for later use i.e. as an injectable therapy.

### EXAMPLE 18

### Sorting for HLA-Patient Matched Fusates in Order to Minimize Graft Rejection

After fusion of a “donor” ES cell and a patient-derived partner cell, the surface antigen expression profile will be a mixture of those proteins expressed from the ES cell and the partner cell. As the fusate proliferates it may lose some or all of one set of chromosomes, with consequent loss of cell surface antigens encoded by those same chromosomes. Thus the cell surface antigen expression may change until the chromosome complement is stable.

One major barrier to tissue transplants is that of rejection. Rejection occurs when the host recognizes the graft as foreign. To minimize this, donor and recipient need to be tissue typed and also blood matched. Rejection based on tissue typing is caused by the recognition of foreign cell surface molecules termed human leukocyte antigens (HLAs). HLA antigens are the MHC class I and II molecules that present antigen to CD8 and CD4 T cells respectively. The HLA molecules are encoded by six major loci that encode structurally homologous proteins: HLA-A, -B, C (MHC class I) and HLA-DR,-DQ and -DP (MEC class II). Together these loci comprise more than 1300 alleles, giving rise to at least 100 different serological specificities.

Rejection by the ABO blood group system is similarly determined by the expression of cell surface markers. The antigens of the ABO system are an integral part of the red cell membrane and of all the cells throughout the body, the plasma of a person contains natural antibodies to A or B, if these antigens are absent from the cells of that person.

If the fusate is to be used for tissue therapy, it should be matched for HLA and ABO, as rejection is dramatically reduced for patients that are tissue typed and blood group matched. However, the cell surface expression of antigens will contain both the donor ES and the patient-derived partner cells HLA/ABO complement, potentially rendering the cells foreign. If chromosomes are lost during growth of the fusate, it is possible that the foreign HLA and/or ABO antigens will be lost as a consequence, rendering the cells compatible with the patient. The cells/tissue obtained from the fusate should therefore be tested for their compatibility for transplantation by tissue typing and ABO blood group matching after the fusate has become genomically stable. Potentially, during the evolution of a stable fusate (i.e. while chromosomes are being lost) compatible cells could be isolated from a mixed population by sorting for the appropriate HLA/ABO groups.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Adorini et al., Springer Semn. Immunopathol. 14: 187-199, 1992
Bach, Endocrine Rev. 15: 516-542, 1994
Harrison, Mol. Med. 1: 722-727, 1995
Honeyman et al., Springer Semin. Immunopathol. 14: 253-274, 1993
Muir et al., Diabetes/Metab. Review 9: 279-287, 1993
Pohl, “Dielectrophoresis”, Cambridge University Press, 1978
Zimmerman et al., Biochimica et Biophysica Acta 641: 160-165, 1981
Zimmerman, Biochimica et Biophysica Acta 694: 227-278, 1982

1. A method for generating mature cells or cells capable of differentiating into mature tissue, said method comprising selecting first and second populations of cells and positioning said first and second populations of cells in a fluid-filled fusing chamber and then subjecting said populations of cells to conditions to facilitate fusion of at least one pair of cells.

2. The method of claim 1 wherein the first and second populations of cells comprise one of a stem cell population and one of a mature cell or stem cells thereof.

3. The method of claim 2 wherein the stem cell is an embryonic stem (ES) cell.

4. The method of claim 2 wherein the stem cell is a neural stem cell.

5. The method of claim 2 wherein the stem cell and the mature cells or its stem cell are selected from the cells in Table 1.

6. The method of claim 1 wherein the fusion conditions are chemical fusion conditions.

7. The method of claim 6 wherein the chemical fusion conditions are polyethylene glycol (PEG) fusion conditions.

8. The method of claim 1 wherein the fusion conditions are electrofusion conditions.

9. The method of claim 6 or 8 wherein the fusion is between two single cells.

10. The method of claim 6 or 8 wherein the fusion is between two populations each of one or more cells.

11. The method of claim 1 wherein the first and second populations of cells are selected using a pipette to extract the first cell population from a group of first cells held in a first container and the second cell population from a group of second cells held in a second container.

12. The method of claim 1 wherein the method comprises:
(a) selecting the first and second cells;
(b) positioning the first and second populations of cells between two electrodes in a fluid filled fusing container, the first and second population of cells being separated from each electrode; and
(c) applying a current having a predetermined waveform to the electrodes to generate a predetermined fusion pulse thereby causing the cells to fuse.

13. The method of claim 12 wherein the cells are held in suspension between the electrodes.

14. The method of claim or 13 further comprising generating a dielectropherisis (DEP) field, the DEP field being adapted to urge the cells towards each other.

15. The method of claim 14 wherein the predetermined waveform comprises a current representing the DEP field.

16. The method of claim 14 wherein the method comprises applying the DEP to a pair of second electrodes.

17. The method of claim 16 wherein the method comprises:
(a) applying a DEP current to the pair of second electrodes;
(b) positioning the first cell in the fusing container, wherein an alternating field is acting to attract the first cell towards one of the second pair of electrodes; and
(c) positioning the second cell in the fusing container, wherein the alternating field is acting to attract the second cell towards the first cell.

18. The method of claim 17 wherein at least one of the first and second populations of cells being positioned in contact with at least one of the second pair of electrodes.

19. The method of claim 12 wherein the method comprises selecting the first and second populations cells using a pipette to extract:
(a) the first cell from a group of first cells held in a first container; and
(b) the second cell from a group of second cells held in a second container.

20. The method of claim 19 wherein the method of positioning the first and second cells in the fusing container comprise:
(a) using the pipette to position the first cell in the fusing container;
(b) using the pipette to position the second cell in the fusing container, adjacent the first cell; and
(c) positioning the electrodes such that the first and second cells are located substantially between the electrodes.

21. The method of claim 19 wherein the pipette is coupled to:
(a) a drive system adapted to move the pipette with respect to the first, second and fusing containers; and
(b) an actuator adapted to actuate the pipette to thereby expel or draw in fluid through a port; where the method comprises using a controller coupled to the drive system and the actuator to move and actuate the pipette.

22. The method of claim 21 further comprising causing the controller to:
(a) move the pipette such that the port is adjacent a cell having predetermined characteristics, the cell being held in fluid suspension in the respective container,
(b) actuate the pipette to draw in fluid through the port, thereby drawing in the cell and the surrounding fluid.

23. The method of claim 21 wherein the method of using the pipette to position the second cell adjacent the first cell comprises causing the controller to:
(a) move the pipette such that the port is adjacent the first cell in the fusing container;
(b) cause the pipette to expel fluid through the port, thereby expelling the second into the fluid in the fusing container;
(c) move the pipette such that the port is as close as possible to both the first and second cells;
(d) cause the pipette to draw in fluid through the port, thereby drawing in the first and second cells and the surrounding fluid;
(e) cause the pipette to expelling the first and second cells into the fluid in the fusing container; and
(f) repeat steps (c) to (e) until the first and second cells are within a predetermined distance.

24. The method of claim 12 wherein the electrodes being coupled to an electrode drive system adapted to move the electrodes with respect to the fusing containers, the method including using a controller coupled to the electrode drive system to position the electrodes in the fusing chamber.

25. The method of claim 12 wherein the electrodes being coupled to a signal generator wherein the method of applying the current to the electrodes comprises causing the signal generator to apply a predetermined current to the electrodes.

26. The method of claim 25 wherein the first and second cells having a respective cell type, the method comprising using a controller coupled to a signal generator to select the current in accordance with the cell types of the first and second cells.

27. The method of claim 26 wherein the first and second cells being the same type of cell and the first and second group of cells being the same group.

28. A method for the treatment or prophylaxis of a subject having mature disease or trauma of an organ or tissue system, said method comprising fusing an ES cell or adult stem cell selected form the list in Table 1 with a mature cell or a stem cell thereof selected from the list in Table 1 to produce a hybrid cell using single cell or bulk cell fusion and then expanding a culture comprising the hybrid cell to a level which is used in tissue replacement and/or rejuvenation therapy.

29. The method of claim 28 wherein the fusion is by single cell electrofusion.

30. A composition comprising a culture of hybrid cells generated by fusing a stem cell and a mature cell or stem cell thereof, said composition further comprising one or more pharmaceutically acceptable carriers and/or diluents.

31. An isolated molecule derived from a hybrid cell generated by the method of claim 1.

32. The molecule of claim 31 wherein the molecule is a cytokine, a heat shock protein, an enzyme, a protein, a ligand or a hormone.
